(19) United States
(12) Patent Application Publication
(10) Pub. No.: US 2021/0378985 A1
(43) Pub. Date: Dec. 9, 2021

(54) METHOD AND COMPOUND FOR THE TREATMENT OF HEPATITIS C

(71) Applicants: Amanuel Tesfazion, Washington, DC (US); Lydia Seifu, Washington, DC (US); Meried Bezuneh, Alexandria, VA (US)

(72) Inventors: Amanuel Tesfazion, Washington, DC (US); Lydia Seifu, Washington, DC (US); Meried Bezuneh, Alexandria, VA (US)

(21) Appl. No.: 17/342,382

(22) Filed: Jun. 8, 2021

Related U.S. Application Data
(60) Provisional application No. 63/036,294, filed on Jun. 8, 2020.

(51) Int. Cl. A61K 31/12; A61P 31/14

(52) U.S. Cl. CPC A61K 31/12; A61P 31/14

(57) ABSTRACT

A method for the treatment of a hepatitis disease, including administering to a subject in need thereof of an anti-pathogenic compound, such that the anti-pathogenic compound is derived from an herbal extract.

### CROSS REFERENCE TO RELATED APPLICATION

This application claims the benefit of, and incorporates by reference, U.S. provisional patent application Ser. No. 63/036,294, entitled “Method and Compound for the Treatment of Hepatitis C,” which was filed on Jun. 8, 2020.

### BACKGROUND

### 1. Field

The present general inventive concept relates generally to a viral disease, and particularly, to a method and compound for the treatment of hepatitis C.

### 2. Description of the Related Art

Hepatitis C is a viral disease caused by the hepatitis C virus (HCV). Viral infection causes liver inflammation, sometimes causing serious liver damage. Infection spreads when blood contaminated with the virus enters the bloodstream of an uninfected person.

The HCV infection may be either acute or chronic. An acute infection is usually undiagnosed because the person is asymptomatic. If symptoms do arise, they often include nausea, vomiting, jaundice, fatigue, fever, muscle or joint pains, and abdominal pain. These symptoms can appear one to three months after exposure and last two to three weeks. Additionally, the acute infection may be resolved on its own.

Unfortunately, chronic infection occurs in nearly eighty percent of cases. Chronic infection may be asymptomatic for many years, while the virus damages the liver until symptoms appear. Chronic infection occurs over many years and results in more serious conditions, including liver failure, cirrhosis (i.e. liver no longer functions due to long term damage), bleeding and bruising easily, fatigue, poor appetite, jaundice, dark-colored urine, itchy skin, swelling in the leg and abdomen, weight loss, spider angiomas, and/or hepatocellular carcinoma (i.e. liver cancer). Cirrhosis substantially increases a person's risk of developing liver cancer.

Hepatitis C is a global disease affecting approximately seventy-one million people worldwide. HCV exists in several distinct genotypes, identified as one through six. HCV Genotype 1b is the most likely to develop cirrhosis. Also, genotypes 1b and 3 are associate with an elevated risk of developing liver cancer.

The following is an excerpt from “Structural Biology of Hepatitis C Virus.” (See https://pubmed.ncbi.nlm.nih.gov/14752815/).

Structural analyses of HCV components provide an essential framework for understanding of the molecular mechanisms of HCV polyprotein processing, RNA replication, and virion assembly. Also, it may contribute to a better understanding of the pathogenesis of hepatitis C. Moreover, these analyses should allow the identification of novel targets for antiviral intervention and development of new strategies to prevent and combat viral hepatitis.

The following is an excerpt from “Binding-Site Identification and Genotypic Profilingof Hepatitis C Virus Polymerase Inhibitors.” (See https://www.ncbi.nlm.nih.gov/pmc/articles/PMC1933266/).

Analysis of HCV polymerase inhibitors have led to identification of several nonnucleoside binding pockets. The shape and nature of binding sites varies due to differences in genotype, which poses challenges to drug development.

To address variability, an HCV mutant and genotypic recombinant polymerase was used to elucidate site of action by profiling with isolates representing genotypes 1a, 1b, 2a, 2b, 3a, 4a, 5a, and 6a.

The control used is a combination of pegylated (i.e. process of both covalent and non-covalent attachment or amalgamation of polyethylene glycol (PEG) polymer chains to molecules and macrostructures, such as a drug, therapeutic protein or vesicle, which is then described as PEGylated) interferon and ribavirin, which has shown effectiveness in only 50% of infected individuals. As such, this testing highlights a weakness and need for new drugs for treatment of people that have failed under current therapy.

The following is an excerpt from “Medicinal plants against hepatitis C virus.” (See https://www.ncbi.nlm.nih.gov/pmc/articles/PMC3961971/).

Commercially available anti-HCV drugs in the market and current approved drugs using a standard of care includes a combination therapy having pegylated interferon alpha (PegIFN) injections and antiviral nucleoside analogue ribavirin (RBV) used for twenty-four to forty-eight weeks, depending upon the type of genotype. Genotype 1 is regarded as most problematic genotype shows the clearance of HCV in 50% of the cases. Similarly, genotype 2 infection shows clearance in only 80% of the cases.

This combination therapy has several considerable side effects such as fever, anemia, flu, and depression. Several combinations of IFN are in clinical trials, such as taribavirin which is a prodrug of ribavirin and albinterferon, which is a combination of IFN alpha and human albumin. The side effects caused by current treatment raised the need to develop antiviral compounds that can suppress or eliminate the infection without toxicity and side effects.

Despite the availability of current treatment, there is a dire need to screen antiviral agents that can target all four genotypes with the same efficacy and without any side effects.

Therefore, there is a need for an effective remedy and/or drug that is natural, inexpensive, and non-toxic. As such, there is a need for a method and compound for the effective treatment of hepatitis C.

### SUMMARY

The present general inventive concept provides method and compound for the treatment of hepatitis C.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a method for the treatment of a hepatitis disease, including administering to a subject in need thereof of an anti-pathogenic compound, such that the anti-pathogenic compound is derived from an herbal extract.

The herbal extract may be a glycol derivative.

The glycol derivative may be diethylene glycol dibenzoate.

The hepatitis disease may be hepatitis C.

The foregoing and/or other features and utilities of the present general inventive concept may also be achieved by providing a method of strengthening a subject infected with a hepatitis disease, including administering to the subject in need thereof of an anti-pathogenic compound, such that the anti-pathogenic compound is derived from an herbal extract.

The herbal extract may be a glycol derivative.

The glycol derivative may be diethylene glycol dibenzoate.

The hepatitis disease may be caused by hepatitis C virus.

The anti-pathogenic compound may cause transversion and cross-links to a lipid-protein coat of the hepatitis C virus to inhibit the hepatitis C virus at entry, such that the hepatitis C virus is prevented from fusing with a plasma membrane of a cell.

The anti-pathogenic compound may prevent replication of the hepatitis C virus by inhibiting NS3 protease.

The anti-pathogenic compound may prevent capsomere assembly during a late-late stage of a life cycle of the hepatitis C virus.

The anti-pathogenic compound may boost an immune system of the subject.

The anti-pathogenic compound may boost the immune system by stimulating production of gamma interferon.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a molecular structure of an anti-pathogenic compound, known as 90I (diethylene glycol dibenzoate), according to an exemplary embodiment of the present general inventive concept;

FIG. 1B illustrates a molecular structure of the anti-pathogenic compound, known as 90I, according to an exemplary embodiment of the present general inventive concept;

FIG. 2 illustrates a graph showing multiple modes of action of 90I in a second part of a 90I study, including AZT and Indinavir;

FIG. 3 illustrates a graph showing late, late mode of action as demonstrated in the second part of the 90I study;

FIG. 4 illustrates an x-ray image PDB on PyMOL of a ligand IDX320 PI and a plurality of residues it interacts with, including H bonds and ID residues;

FIG. 5 illustrates 90I interacting with five H bonds on SER 136, GLY 137, SER 138, and SER 139;

FIG. 6 illustrates another pose of 90I with four H bonds on SER 136, GLY 137, SER 138, and SER 139;

FIG. 7 illustrates a binding pocket view of 90I disposed within a binding pocket interacting with five H bonds on SER 136, GLY 137, SER 138, and SER 139 ALL;

FIG. 8 illustrates 90I disposed within the binding pocket interacting with SER 136, GLY 137, SER 138, and SER 139;

FIG. 9 illustrates another pose of 90I interacting with SER 139, SER 138, LYS 135, and ALA157 with four H bonds;

FIG. 10 illustrates 90I with additional H bonds interacting with residues;

FIG. 11 illustrates 90I with additional H bond interactions with residues;

FIG. 12 illustrates 90I interacting with -LYS 136, ALA157, and HIS 57, including three H bonds;

FIG. 13A illustrates 90I interacting with THR 10 and ARG 11;

FIG. 13B illustrates a pocket view of 90I interacting with THR 10 and ARG 11;

FIG. 13C illustrates another pocket view of 90I interacting with THR 10 and ARG 11;

FIG. 14 illustrates 90I interacting with GLN 34 and ARG 11 including two H bonds;

FIG. 15 illustrates 90I interacting with GLN 34 and GLU 30;

FIG. 16 illustrates 2D structures of lead compounds to acts as novel, potent, and structurally diverse inhibitors of HCV NS3/4A protease;

FIG. 17 illustrates an x-ray image of 4a92 PDB with PI from PDB including a ligand in orange on protein with H bond in yellow and residues THR 160 and HIS 528 in blue;

FIG. 18 illustrates a second pose of 90I interacting with HIS 528;

FIG. 19A illustrates 90I interacting with residues THR 160 and GLY 162;

FIG. 19B illustrates another pose of 90I interacting with residues THR 160 and GLY 162;

FIG. 20A illustrates another pose of 90I interacting with residue HIS 528 with an H bond;

FIG. 20B illustrates another pose of 90I interacting with residue HIS 528;

FIG. 20C illustrates another pose of 90I interacting with residue HIS 528;

FIG. 21 illustrates 90I Interacting with residue SER including three H bonds;

FIG. 22 illustrates 90i disposed within a binding pocket;

FIG. 23A illustrates 90I disposed within a binding pocket while interacting with residue HIS 528;

FIG. 23B illustrates another view of 90I disposed within the binding pocket;

FIG. 23C illustrates a different view of 90I disposed within the binding pocket;

FIG. 23D illustrates 90I disposed within the binding pocket interacting with HIS 528;

FIG. 24 illustrates 90I disposed within a binding pocket interacting with at least one residue and creating an H bond thereto;

FIG. 25 illustrates 90I disposed within a binding pocket interacting residue GLY 162 including two H bonds;

FIG. 26 illustrates 90I disposed within a binding pocket interacting with residues, GLY 162 and THR 160;

FIG. 27 illustrates 90I disposed within a binding pocket with four H bonds;

FIG. 28 illustrates 90I disposed deep within a binding pocket;

FIG. 29 illustrates a book excerpt regarding residues;

FIG. 30 illustrates 90I interacting with residues THR 295, SER 294, SER 459, and GLN 460 including four H bonds;

FIG. 31 illustrates a possible HPI binding site on NS3;

FIG. 32 illustrates an inhibitor 1 bound to a HCV NS4/4A;

FIG. 33 illustrates a schematic representation of the HCV NS3/4A protease;

FIG. 34A illustrates a first pose of 90I disposed within a binding pocket with four H bonds;

FIG. 34B illustrates another view of the first pose of 90I disposed within the binding pocket with four H bonds;

FIG. 34C illustrates a 12 A zoomed in first surface view of the first pose of 90I disposed within the binding pocket;

FIG. 34D illustrates another zoomed in full surface view of the first pose of 90I disposed within the binding pocket;

FIG. 34E illustrates another zoomed in view of 90I disposed within the binding pocket interacting with an H bond;

FIG. 35A illustrates a different pose of 90I interacting with a residue HIS 528;

FIG. 35B illustrates 90I interacting with the residue HIS 528;

FIG. 36 illustrates 90I interacting with residues HIS 528 and GLN 526 including three H bonds;

FIG. 37A illustrates 90I interacting with polymerase inhibitors, glecaprevir and pibrentasvir, as controls;

FIG. 37B illustrates 90I in white disposed on polymerase and a plurality of binding pockets interacting with polymerase inhibitors, glecaprevir and pibrentasvir;

FIG. 37C illustrates 90I in white disposed on polymerase and the plurality of binding pockets interacting with polymerase inhibitors, glecaprevir and pibrentasvir; and

FIG. 37D illustrates 90I in white disposed on polymerase and within at least one binding pocket interacting with polymerase inhibitors, glecaprevir and pibrentasvir.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

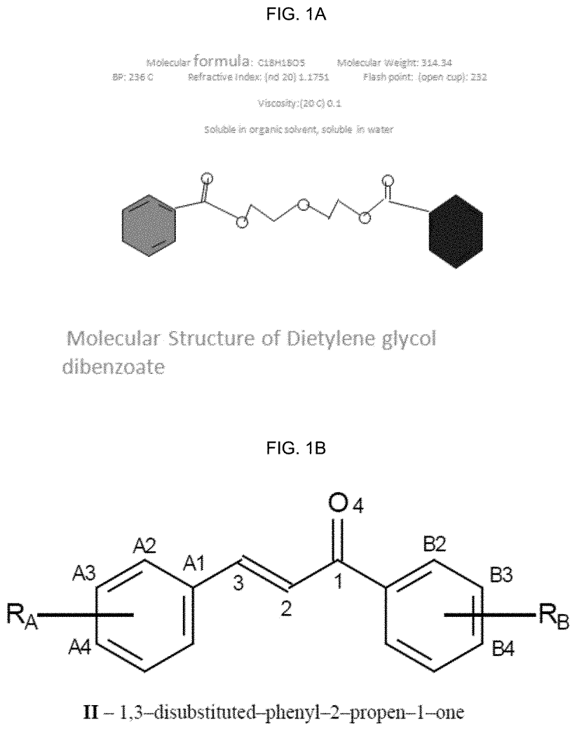

FIG. 1A illustrates a molecular structure of an anti-pathogenic compound, known as 90I (diethylene glycol dibenzoate), according to an exemplary embodiment of the present general inventive concept.

FIG. 1B illustrates a molecular structure of the anti-pathogenic compound, known as 90I, according to an exemplary embodiment of the present general inventive concept.

A highly active antireroviral drug will be tested. The highly active antiretroviral drug is based on a novel molecule that has been discovered within an herb located in Ethiopia that may be used to treat subjects (e.g., people, animals, etc.) infected with a pathogen, which in this case is hepatitis C. However, although treatment is the term used, treatment may also include prophylaxis (i.e. prevention) and/or vaccination, such that the treatment may inhibit replication of and/or kill the pathogen. The observed window of efficacy at a traditional herbal treatment center in Ethiopia, used in treating AIDS patients, served as the basis for undertaking the developmental investigation of the crude product for further drug development investigation.

Referring to FIGS. 1A and 1B, an anti-pathogenic agent and/or the anti-pathogenic compound may be identified as 90I (or 90i). The anti-pathogenic compound may be derived from the herbal extract identified as H2K1001. H2K1001 and/or 90I may be a natural product that was isolated using the Bioassay Guided Fractionation, and further purified, molecularly characterized (i.e. characterizing at the molecular level without any effect of environment or development or physiological state of the organism), and not only found to be highly potent against all human immunodeficiency virus (HIV and/or HIV-1) strains, but also immunogenic with unique multiple modes of action (i.e. functional or anatomical change at a cellular level, resulting from exposure of a living organism to a substance), potently effective against both reverse transcriptase and a protease (PR) enzyme (i.e. an enzyme which breaks down proteins and peptides). The essence of combination drug therapy, HAART regiments, may be its effectiveness against all HIV-1 strains that is potent enough to bring the viral load down to an undetectable level. This may be achieved by combining an RT and a PR combination synergy to affect multiple modes of action.

Furthermore, although the pathogen is identified as hepatitis C, 90I may be used to treat any pathogen including a virus, bacteria, protozoan (i.e. parasite), and/or fungal.

Bacterial pathogens may include Mycobacterium tuberculosis Tuberculosis, Bacillus anthracis Anthrax, and Staphylococcus Sepsis aureus, but is not limited thereto.

Viral pathogens may include Adenoviridae, Mastadenovirus, Infectious canine hepatitis, Arenaviridae, Arenavirus, Lymphocytic choriomeningitis, Caliciviridae, Norovirus, Norwalk virus infection, Coronaviridae, Coronavirus, Severe Acute Respiratory Syndrome, SARS-CoV, SARS-CoV-2, Torovirus, Filoviridae, Marburgvirus, Viral hemorrhagic fevers, Ebolavirus, Viral hemorrhagic fevers, Flaviviridae, Flavivirus, West Nile Encephalitis, Hepacivirus, Hepatitis C virus infection, Pestivirus, Bovine Virus Diarrhea, Classical swine fever, Hepadnaviridae, Orthohepadnavirus, Hepatitis, Herpesviridae, Simplexvirus, cold sores, genital herpes, bovine mammillitis, Varicellovirus, chickenpox, shingles, abortion in horses, encephalitis in cattle, Cytomegalovirus, infectious mononucleosis, Mardivirus, Marek's disease, Orthomyxoviridae, Influenzavirus A, Influenza, Influenzavirus B, Influenza, Papillomaviridae, Papillomavirus, Skin warts, skin cancer, cervical cancer, Picornaviridae, Enterovirus, Polio, Rhinovirus, Common cold; Aphthovirus, Foot-and-mouth disease, Hepatovirus, Hepatitis, Poxviridae, Orthopoxvirus, Cowpox, vaccinia, smallpox, Reoviridae, Rotaviruses, Diarrhea, Orbivirus, Blue tongue disease, Retroviridae Gammaretrovirus, Feline leukemia, Deltaretrovirus, Bovine leukemia, Lentivirus, Human immunodeficiency, FIV, and SIV, Rhabdoviridae, Lyssavirus, Rabies, Ephemerovirus, Bovine ephemeral fever, Togaviridae, Alphavirus, and Eastern and Western equine encephalitis, but is not limited thereto.

Parasitic pathogens may include Plasmodium, Malaria, Leishmania, and Leishmaniasis, but is not limited thereto.

Fungal pathogens may include Aspergillis, Candida, Coccidia, Cryptococci, Geotricha, Histoplasma, Microsporidia, and Pneumocystis, but is not limited thereto.

As such, 90I may also be an anti-pathogenic compound that is applicable to different diseases and/or infections.

The Ethiopian region may be characterized by a wide range of ecological, edaphic, and climatic conditions that account for the wide diversity of its biological resources, both in terms of flora and faunal wealth. The plant genetic resources of the country exhibit an enormous diversity as seen in the fact that Ethiopia is one of the twelve Vavilov Centers of origin for domesticated crops and their wild and weedy relatives. According to recent studies, it is estimated that there are more than seven thousand species of flowering plants recorded in Ethiopia, of which at least twelve percent are probably endemic.

Medicinal plants may comprise one of the important components of Ethiopian vegetation. On record, there may be six hundred species of medicinal plants constituting a little over ten percent of Ethiopia's vascular flora. The medicinal plants may be distributed all over the country, with greater concentration in the south and southwestern parts of the country. Woodlands of Ethiopia may be the source of most of the medicinal plants, followed by the montane grassland and/or dry montane forest complex of the plateau. Other important vegetation types for medicinal plants may be the evergreen bushland and rocky areas.

As such, an herbal extract may be extracted from the herb from Ethiopia. The herbal extract may include a glycol derivative. Moreover, the glycol derivative may include diethylene glycol dibenzoate. An anti-pathogenic compound may include diethylene glycol dibenzoate to treat hepatitis C.

The following is another excerpt from “Medicinal plants against hepatitis C virus.” (See https://www.ncbi.nlm.nih.gov/pmc/articles/PMC3961971/).

All viruses start their life cycle through attachment and entry into host cell and then increase their progeny by transcription and replication of the genome. The RNA viruses such as influenza, HIV, and HCV have become a matter of concern as these are highly variable and lack an RNA dependent RNA polymerase proofreading mechanism.

Infectious diseases have widely been treated using medicinal plants and twenty-five percent of current medicines have compounds from medicinal plants. There are plenty of plants that are known for their magical medicinal properties and these plants serve can serve as important reservoir for drug discovery against infectious disease.

Liver diseases have been treated around the world using numerous medicinal plants and their formulations and this has given confidence to researchers to investigate the effect of these medicinal plants against HCV in more depth.

Medicinal plant phytochemicals as anti-HCV agents:

Diosgenin is a plant derived sapogenin that has effectively blocked the replication of the HCV subgenomic replicon at both mRNA (i.e. messenger RNA that corresponds to the genetic sequence of a gene and is read by the ribosome in the process of producing a protein) and protein level.

Silymarin, which isolated from Silybum marianum has been tested against HC core protein of genotype 3a and is found to be effective in inhibiting the viral core expression.

Lamium album has blocked HCV entry with the CD81 (i.e. Cluster of Differentiation 81, a protein in humans that plays a role in binding to the hepatitis E2 glycoprotein dimer) receptor.

Naringenin, a predominant flavanone (i.e. a type of a class of polyphenolic plant metabolites, which is a ketone that occurs in plants) in grapefruit, has suppressed the activity of core protein in Huh 7 (i.e. a type of human liver cell that may be grown in a laboratory for research purposes) cells and has also effectively blocked the assembly of HCV particles.

90I as an Anti-HCV

Based on some of the existing plant phytochemicals identified above, 90I may be a perfect candidate for ant-HCV study due to its multiple modes of action in both early and late stages of the HIV life cycle, highly active protease inhibitor, unique molecular property, anti-oxidant, non-toxicity, and a byproduct of medicinal plant. HCV establishes chronic infections in approximately three percent of the world's population. Infection leads to progressive liver disease and hepatocytes are the major site of viral replication in-vivo. However, chronic infection is associated with a variety of extrahepatic syndromes, including central nervous system (CNS) abnormalities. A series of neural and brain-derived cell lines are screened for their ability to support HCV entry and replication.

HIV-1 in vitro data analysis of 90I, via Bioassay Guided Fractionation, may confirm observed in vivo data and was used to isolate a lead molecule.

Full blown in vitro studies may demonstrate astonishing results that surpass any known commercially available antiretroviral (ARV) and/or highly active antiretroviral therapy (HAART) drugs currently on the market.

Due to 90I demonstrating high effectiveness against HIV with multiple modes of action, 90I may provide similar effectiveness to inhibit HCV. Furthermore, HIV and HCV share several molecular cross-talks in their life cycle and this is the basis for initiating 90I evaluation against HCV.

90I has proven to stimulate the production of gamma interferon (i.e. a dimerized soluble cytokine that is the only member of the type II class of interferons and is a product of human leukocytes and human lymphocytes). More specifically, 90I may migrate from blood into tissue and differentiate into tissue macrophages, such that each of the tissue macrophages may serve as a vehicle for transporting viruses to a variety of tissues. The ability of antiretroviral agents to cross the blood brain barrier is one important consideration, since the brain acts as a sanctuary for viruses as well as a site for disease progression.

This assay may underscore 90I's absorptions in a monocyte and/or a macrophage primary cell, such that the anti-pathogenic compound may have stability and longer pharmacokinetic half-life in ten days assay with single time point drug addition. Another significance of this result is that 90I and/or HK1001 may reinstate a dysfunctional monocyte to resume its natural functional role as a primary effector cell in the cellular immune system, effecting extensive anti-microbial and/or anti-fungal functional capability in the killing of multiple pathogens and/or other opportunistic infecting agents, such as hepatitis C.

Moreover, activated CD8+ cells are reported to produce high levels of gamma interferon, which may be involved in the anti-HIV-1 immune responses, contributing to both control of viral spread and concomitant lymphoid follicular lyses. An amount of gamma interferon produced by 90I may be equivalent to that of the positive control, PMA-lonomycin combination. The conclusion from this result may be that 90I stimulates cellular genes to produce gamma interferon. This finding may have a far-reaching implication and relevant to interleukin 12 (IL-12) (i.e. a cytokine that is produced by activated antigen-presenting cells, such as dendritic cells and/or macrophages). The Th-2 subset may favor a humoral response, including IL-4, IL-5, and IL-6 and causes activation of B cells (i.e. B lymphocytes) leading to antibody formations.

Furthermore, Th1 (i.e. a subset of T lymphocytes that express CD4 and are known as T helper cells, they produce cytokines, specifically Th1-type cytokines) provides strong immunological response, whereas Th2 is associated with progression of HIV-1 pathogenesis. H2K1001 and/or 90I effecting gamma interferon production may be the result of Th1 subset boosting, which could have a far greater impact on reversing the course of HIV-1 infection. This study shows that 90I is not only a potent antiviral, but also an immune system booster.

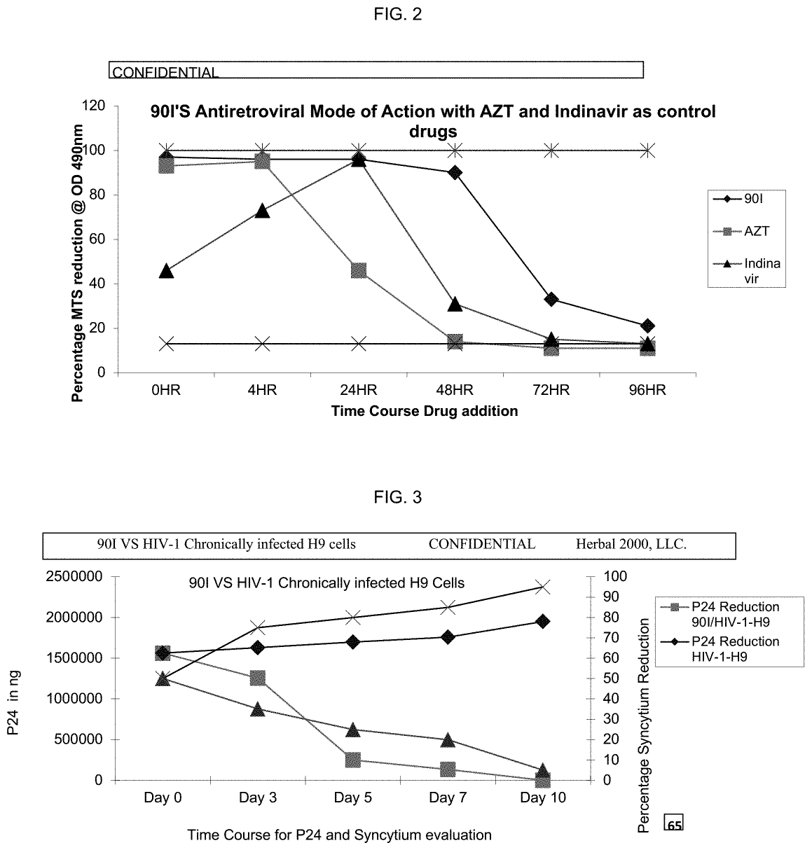

FIG. 2 illustrates a graph showing multiple modes of action of 90I in a second part of a study, including AZT and Indinavir.

Referring to FIG. 2, 90I, azidothymidine (AZT) (i.e. a nucleoside reverse transcriptase inhibitor or NRTI), and Indinavir (i.e. a protease enzyme inhibitor of HIV or PRI) were included to determine the functionality of a time course study. As the dose response curve illustrates H2K1001 and/or 90I may inhibit HIV-1 both early and late. The mechanism and/or modes of action by which H2K1001 and/or 90I may inhibit HIV-1 is defined by the evaluation of a cell-based time course study and assessments of each antiretroviral study conducted for H2K1001 and/or 90I. The results have been demonstrated both in phase I and phase II studies. The time course study may be an important tool for the determination of viral kinetics and drug mode of action. The dynamics of drug-virus interaction, competitive or non-competitive, kinetics provides a dose response curve from which a broad lead mechanism of action can be drawn. The dynamics of the interactions relate to the viral kinetic of infection and drug addition at various time points. In the various cell-based assays conducted on H2K1001 and/or 90I, it may be reasonably concluded that the compound works both early and late.

FIG. 3 illustrates a graph showing late, late mode of action as demonstrated in the second part of the 90I study.

Referring to FIG. 3, H2K1001 and/or 90I is compared to untreated controls, yet effectively blocked the progression of HIV-1 infection. The co-cultivation study results with H2K1001 and/or 90I demonstrating the following: (a) a reversal of viral burden from 1.7×10\hat{ }6 pg of p24 to 5×10\hat{ }2, (b) a drastic drop of syncytium formation, and (c) exponential cell growth dynamics with greater than ninety percent cell viability. These results demonstrate may highlight other significant properties of H2K1001 and/or 90I. Viral pathogenesis in co-cultivation of infected cells with uninfected target cells presents a complex system with several target sites (attachment, fusion, transcription, processing, packaging and budding).

In many documented antiretroviral studies, the co-culture system is conventionally used to isolate the virus as well as to study cell-to-cell transmission or to determine if a given compound blocks cell fusion. H2K1001 and/or 90I may use aggressive intervention that could possibly be directed to at least one of a late phase, early fusion and/or attachment, and both early and late. However, judging from the variety of 90I results, early and late mode of action is consistent to this compound. This study has shown that 90I has effectively blocked the progression of latent HIV-1 from cross transmitting infection to the uninfected cells, reversed the latent infected cells from the course of HIV-1 pathogenesis or apoptosis, and/or demonstrated early and late mode of action.

As shown above, 90I includes multiple modes of action, inhibition of early and/or late at protease, and inhibition during late-late at capsomere (i.e. a subunit of a capsid, which is the protein shell of a virus, that is an outer covering of protein that protects the genetic material of a virus) assemblage targets of HIV life cycle.

Molecular Intervention of HCV by 90I

HCV typically attaches to and infects liver cells in order to carry out its life cycle and reproduce, which is why it is associated with liver disease. While various details remain unknown about the exact natural processes of hepatitis C, like other viruses, it must complete key steps to carry out its life cycle:

(1) The virus locates and attaches itself to a liver cell. Hepatitis C uses particular proteins present on its protective lipid coat to attach to a receptor site (i.e. a recognizable structure to attach to on the surface of a cell, such as a liver cell).

A first target of 90I against hepatitis C is because 90I may be rich in epoxy oxygen capable of transversion (i.e. a point mutation in deoxyribonucleic acid (DNA), where a single purine is changed for a pyrimidine, or vice versa; a transversion can be spontaneous, or can be caused by ionizing radiation or alkylating agents) and cross linking to a lipid-protein coat inhibiting HCV at entry level. As such, 90I may preventing HCV from fusing with a plasma membrane.

(2) The virus' protein core penetrates the plasma membrane and enters the cell. To accomplish this, hepatitis C utilizes its protective lipid (fatty) coat, merging its lipid coat with a cell's outer membrane (i.e. the coat is composed of a fragment of another liver cell's plasma membrane). Once the lipid coat has successfully fused to the plasma membrane, the membrane engulfs the virus and the viral genetic material is inside the cell.

(3) The protein coat dissolves to release the viral RNA in the cell. This may be accomplished during penetration of the cell membrane (i.e. it is broken open when it is released into the cytoplasm), or special enzymes present in liver cells may be used to dissolve the casing.

(4) The viral RNA then corrupts the cell's ribosomes and begins the production of materials necessary for viral reproduction. Because hepatitis C stores its information in a “sense” strand of RNA, the viral RNA itself can be directly read by the host cell's ribosomes and function like the normal RNA present in the cell. As it begins producing the materials coded in its RNA, the virus also possibly shuts down most of the normal functions of the cell, such that it conserves energy for the production of viral material.

Although, it occasionally appears that hepatitis C will stimulate the cell to reproduce (i.e presumably to create more cells that can produce viruses), which is why hepatitis C is often associated with liver cancer. The viral RNA first synthesizes the RNA transcriptase it will need for reproduction.

Once there is adequate RNA transcriptase, the viral RNA creates an antisense version (i.e. a paired opposite) of itself as a template for the creation of new viral RNA. As such, the viral RNA is now copied hundreds or thousands of times, making the genetic material for new viruses. Some of this new RNA will contain mutations.

A second target of 90I may include the RNA transcriptase, the key molecule for hepatitis C replication in the liver. As discussed above, 90I may be a strong protease inhibitor that could completely knockout the HCV NS3 protease (i.e. a nonstructural protein of HCV that is a serine protease and is responsible for cleavage at four sites of the HCV polyprotein). As such, 90I may interfere with the replication of HCV genome and restores the pathway of innate immunity.

(5) Viral RNA then directs the production of protein-based capsomeres (i.e. the building blocks for the virus's protective protein coat). Ribosomes create the proteins and release them for use.

A third target for 90I may include capsomere assembly. As discussed above, 90I is highly effective at late-late stage, such that 90I may inhibit at the capsomere assembly stage of HCV life cycle.

The fundamental underlying advantage that 90I has in comparison to the current treatments used for hepatitis C may include a flavonoid phytochemical effective anti-oxidant that may prevent liver cancer, multiple molecular modes of action that parallels to not one, but all currently used treatments (i.e. protease inhibitors and interferon producer), multiple natural lead isolates identified, multiple modes of application, highly active (HAART), proven effective against resistance, such that promoting use of this drug without the need of combinatorial drugs being required, a natural product, provides a boost to the immune system, reverse latent infection, highly effective in brain cells, non-toxic, and affordable, but is not limited thereto.

Based on evidence presented in in-vitro studies, and as discussed above, 90I has proven to be (1) rich in epoxy oxygen capable of transversion and cross linking to a lipid-protein coat inhibiting HCV at entry level and may prevent HCV fusing with a plasma membrane, (2) inhibition of RNA transcriptase, the key molecule for hepatitis C multiplication in the liver, by inhibiting HCV NS3 protease, and (3) inhibition of capsomere assembly. As such, this evidence prompted further investigation through use of Auto Dock.

Molecular Intervention of HCV by 90I

Protease and Polymerase Inhibition Against HCV by 90I

Associating 90I with HCV protease inhibition by a method of identifying residues that are located on HCV protease or polymerase. 90I not only interacts with the binding sites and pockets, it may interact with sites an allosteric (i.e. of, relating to, undergoing, or being a change in the shape and activity of a protein (such as an enzyme) that results from combination with another substance at a point other than the chemically active site) drug would interact outside the binding domain. 90I may interact with many residues and show lots of hydrogen bond (H bond) interaction Based on research, conclusions, testing, and experimenting, 90I may absolutely be used as a treatment for HCV.

90I has already shown a high Therapeutic Index, excellent fifty percent effective concentration (EC50), and fifty percent inhibitory concentration IC50 and does work in vitro against HIV. As such, 90I may have a biological significance as shown in-vitro against HIV with its high therapeutic index. Consequently, the drug will go through a toxicity test for IND application and soon proceed with clinical trial.

To determine if this same molecule with biological significance will also be a good candidate against HCV an in-silico study has been performed as shown below.

Subsequently, 90I should be given a chance to proceed in-vitro and then clinical trial.

As discussed above, 90I may inhibit protease and also polymerase enzymes.

Specifically, 90I may act as three types of polymerase inhibitors. Substrate analogs (nucleoside and nucleotide analogs), allosteric inhibitors (non-nucleoside inhibitors), and inhibitors that intercalate (i.e. insert something between layers in a structure) or directly interact with nucleic acids as a non-nucleoside reverse transcriptase inhibitor (NNRTI) and non-covalent.

As discussed above, the HCV NS3 protein is essential for viral polyprotein processing and RNA replication and hence viral replication. It is composed of an N-terminal serine protease domain and a C-terminal helicase/NTPase domain. HCV NS3/4A protease is a prime target for developing direct-acting antiviral agents.

The NS3-4A serine protease is responsible for the proteolytic cleavage (i.e. the breakdown of proteins or peptides into amino acids by the action of enzymes) at four junctions of the HCV polyprotein precursor:

Macrocyclic Hepatitis C Virus NS3/4A Protease Inhibitors

An Overview of Medicinal Chemistry. The 9.6 kb RNA genome of HCV encodes approximately 3000 amino acid residues of its polyprotein that must be processed by host and viral proteases into both structural (S) and non-structural (NS) proteins, respectively.

Additional research that has been done regarding HCV are shown below.

The following is an excerpt from “Macrocyclic Hepatitis C Virus NS3/4A Protease Inhibitors: An Overview of Medicinal Chemistry.” (See https://pubmed.ncbi.nlm.nih.gov/27160539/).

HCV is a causative agent of hepatitis C infectious disease that primarily affects the liver, ranging in severity from a mild illness lasting a few weeks to a lifelong illness. The 9.6 kb RNA genome of HCV encodes approximately 3000 amino acid polyprotein that must be processed by host and viral proteases into both structural (S) and non-structural (NS) proteins, respectively. Targeting the serine protease NS3 with an activating factor NS4A, which has been considered as one of the most attractive targets for the development of anti-HCV therapy. Although, there is no vaccine available, antiviral medicines cure approximately 90% of the persons with hepatitis C infection. On the other hand, efficacy of these medications can be hampered due to the rapid drug and cross resistances. To date, all developed HCV NS3/4A inhibitors are mainly peptide-based compounds derived from the cleavage products of substrate. Specifically, macrocyclic (i.e. relating to or denoting a ring composed of a relatively large number of atoms, such as occurs in heme, chlorophyll, and several natural antibiotics) peptidomimetics (i.e. a small protein-like chain designed to mimic a peptide) have rapidly emerged as a classical NS3/4A protease inhibitors for treating the HCV infection.

However, 90I is a small molecule and much less complex than peptides. As such, 90I may cause a less toxic reaction and have high efficacy due to it being smaller.

When observing 90I in-silico, it may interact with most of the residues mentioned in the research, similar to the much larger peptide macrocyclic inhibitors. Moreover, 90I may accomplish what a larger peptide molecule can (e.g., inhibiting protease). Since the larger molecule was tested in-vitro and did go to clinical trial, the larger molecule was compared to 90I with belief that it will be less toxic and yet interact with said residues to effectively have a good efficacy and IC value.

A study was performed using a macrocyclic inhibitor IDX320, since it is an actual experiment as described below.

The following is an excerpt from “Journal of Hepatology, In Vitro Antiviral Activity of IDX320, A Novel and Potent Macrocyclic HCV Protease Inhibitor.” (See https://www.journal-of-hepatology.eu/article/S0168-8278(10)60770-2/pdf).

Background and Aims: This study evaluated the in-vitro antiviral activity of IDX320, a novel macrocyclic inhibitor of HCV protease, in biochemical and replicon assays.

Methods: The antiviral activity and specificity of IDX320 were evaluated in a variety of standard assays utilizing purified proteases, HCV replicons, and an infectious HCV virus. The resistance profile of IDX320 was determined in replicon selection experiments as well as transient transfection assays using site-directed mutant replicons.

Results: IDX320 is a potent non-covalent inhibitor of HCV protease enzymes of genotypes 1a, 1b, 2a, and 4a (0.8 to 1.9 nM IC50) and genotype 3a (23 nM IC50). In surface plasmon resonance studies, IDX320 bound tightly to NS3/4A protease (KD of 0.8 nM) with a dissociation half-life of >9 hours. Nine human cellular proteases were not inhibited by IDX320 (IC50>15 mM). In cellular assays, IDX320 inhibited genotype 1b replicons with sub-nanomolar potency (EC50 0.5 nM; SI 50,400), genotype 1a replicons (EC50 3.4 nM; SI>22,985) and genotype 2a JFH-1 virus (EC50 4.7 nM; SI 2,568). Treatment of replicon cells for 14 days produced dose-dependent reductions in replicon RNA levels, with a maximum reduction of 3.7 log 10 at 10 nM IDX320. The NS3 D168V mutation was the signature resistance mutation, selected in all IDX320-resistant replicon cell lines. Studies on replicons bearing site directed protease resistance mutations indicated that Q80R, R155K, A156T, or D168A/E/V/Y conferred resistance to IDX320, while T54A, R155Q and A156S mutants remained susceptible. Replicons bearing the D168V mutation as well as the IDX320-resistant cell lines remained fully susceptible to IFN plus ribavirin, and direct-acting antivirals of different classes.

Conclusion: IDX320 is a potent inhibitor of HCV NS3/4A protease and HCV replication in cell culture with broad genotypic coverage. In-vitro selectivity was demonstrated against several human cellular proteases and cell lines. IDX320 bound tightly to HCV protease with a long dissociation half-life. These favorable in-vitro characteristics, along with others presented by Good et al. 1, support the evaluation of IDX320 in the clinic.

Reference(s) [1] Good et al, “Preclinical pharmacokinetic profile of IDX320, a novel and potent HCV protease inhibitor”; (submitted EASL 2010).

Here, we see that IDX320 has gone all the way to clinical trial which means it has and/or had hope.

The following is an excerpt from “Safety and clinical effects of IDX320 in Hepatitis C infection.” (See https://doi.org/10.1186/ISRCTN44746369).

Also, the following article discloses a clinical trial of a macrocycle PI inhibitor, which is a candidate for use as a control/reference drug in order to compare it to 90I.

The following is an excerpt from “Idenix Pharmaceuticals Research and Development Update on HCV Programs, Phase 1/11: IDX320, an HCV protease inhibitor.” (See http://www.natap.org/2010/HCV/072710_01.htm).

In the second quarter of 2010, Idenix initiated a three-day proof-of-concept study in 38 treatment-naive HCV genotype 1-infected patients. This trial is a Phase 1/11 randomized, parallel-arm, double-blind, placebo-controlled study evaluating the safety and antiviral activity of IDX320. The study is evaluating four doses of IDX320, ranging from 50 to 400 mg once-per-day, and one 200 mg twice-daily dose. Data from this study will be submitted as a late-breaker to the upcoming AASLD meeting.

IDX320 Testing

From in-vitro to in-silico by using an actual x-ray crystalized image of IDX320 bound with NS3/4A serine protease. Also, x-ray was followed by in-silico testing for quality control.

An x-ray image from Protein Data Bank (PDB) was used to show an actual ligand IDX320 interacting with binding residues of the hepatitis C virus NS3/4A serine protease using PyMOL (i.e. an open source molecular visualization system). The residues used are as follows and agree with other studies: LEU 135, LYS136, GLY137, SER138, SER139, ALA156, ALA157.

HCV NS3/4A serine protease in complex with 6570 (See https://www.rcsb.org/structure/4u01). 4U01 PDB was used with its crystalized structure with inhibitor.

Method: X-RAY DIFFRACTION.

Discovery and structural diversity of the hepatitis C virus NS3/4A serine protease inhibitor series leading to clinical candidate IDX320.

Initially, ligand IDX320 was removed from the protein after making note of the residues from the x-ray image. Subsequently, 90I was run on autodock to observe its interaction with the same residues.

The log files shown below are for IDX320 and 90I using a similar method of a ‘seek and identify’ algorithm, since the ‘seed’ used is the same. The seed is autodock vina seed number. This makes the comparison of 90I with IDX320 a correct one. A random seed for IDX320 was used, followed by the same seed that was used in IDX320 and applied to 90I. Random seeds were also used to test, (this method is different than using same autodock seed) with both yielding the same results. The test conducted was accurate in predicting the interaction. Specifically, 90I was ran on HIV in-silico to make sure autodock was accurately predicting the interactions. 90I TI is 5000 in-vitro when tested on HIV. Therefore, autodock does predict the interactions rather well.

Referring to Table 1, the log files include 90I on left and IDX320 on right. The images corresponding to the log files can be seen below.

Notably, the 90I molecule is much smaller than a IDX320 peptide molecule and yet 90I has acceptable affinity and lots of good H bond distance as root-mean-square deviation (RMSD) values. The overall interpretation is that 90I will inhibit HCV protease.

IDX320 shows to have −15 kcal/mol, and H bond of less than 3 Argon (RSMD) to be an ideal distance from the residue atom that interacts to create H bond. Anything less than −5 kcal/mol is acceptable.

According to previous studies found in peer-reviewed literature, there may be more significance of binding with a protein-ligand complex and having the lowest energy, such that there is a better binding affinity. Moreover, a benchmark being −5 kcal/mol or less is better.

IDX320 is a larger molecule with more affinity using less energy measured in kcal/mol for the energy used for binding. As stated above, the lesser the better. IDX320 shows −15 kcal/mol which is less than 90I meaning that it consumes less energy, but 90I is a smaller molecule with TI higher than 5000 and is less toxic.

As mentioned previously, a binding with a protein-ligand complex and having the lowest energy, results in a better binding affinity. The benchmark is 5 kcal/mol or less is better, and an H bond of less than 3 Argon RMSD to be an ideal distance from the residue atom that interacts to create an H bond. There are eight 90I poses that have polar H bond interactions with less than 3 Argon RMSD distance. 90I H bonds are interacting with the residues binding domain affecting HCV protease from functioning properly.

If short distance H bonds are not found, and yet the molecule shows 2 or more H bonds, this can be interpreted as stable binding during different poses. The affinity for 90I is almost always −7 kcal/mol which is desirable.

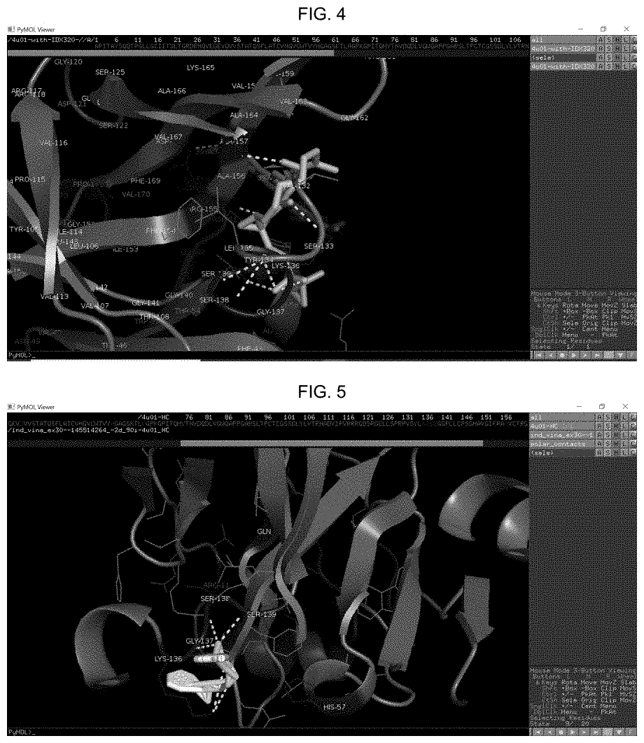

FIG. 4 illustrates an x-ray image PDB on PyMOL of a ligand IDX320 PI and a plurality of residues it interacts with, including H bonds and residue ID's.

Referring to FIG. 4, the residues are similar to other studies included below, such as LYS 136 GLY 137 SER 138 AND SER 139 that are identified using PyMOL from an x-ray digitized image. The protein is 4u01 PDB, as described above.

FIG. 5 illustrates 90I interacting with five H bonds on LYS 136, GLY 137, SER 138, and SER 139.(one being double bond with two different 90i atoms)

Referring to FIG. 5, even at a far distance, 90I has four H bond interactions making it stable.

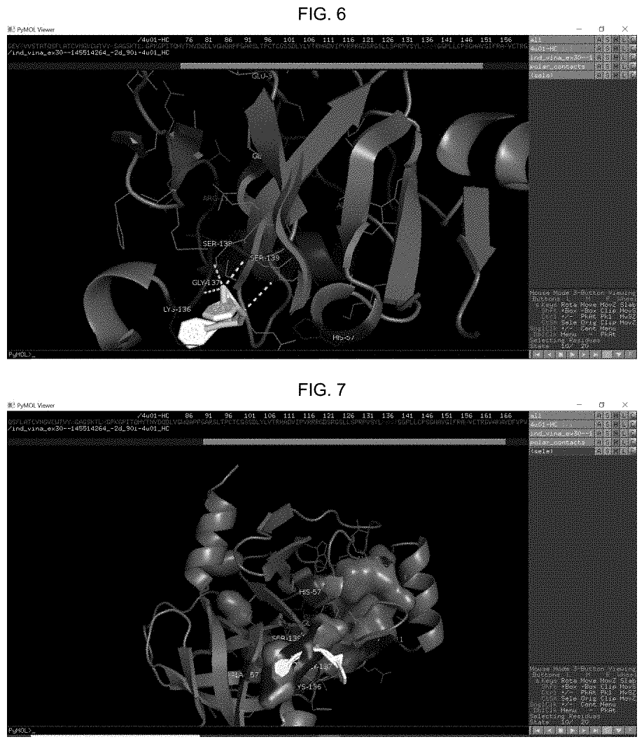

FIG. 6 illustrates another pose of 90I with four H bonds on LYS 136, GLY 137, SER 138, and SER 139.

Referring to FIG. 6, even at the far distance, the another pose of 90I has four H bonds interactions making it stable.

FIG. 7 illustrates a binding pocket view of 90I disposed within a binding pocket interacting with five H bonds on LYS 136, GLY 137, SER 138, and SER 139 ALL (one being double bond with two different 90i atoms).

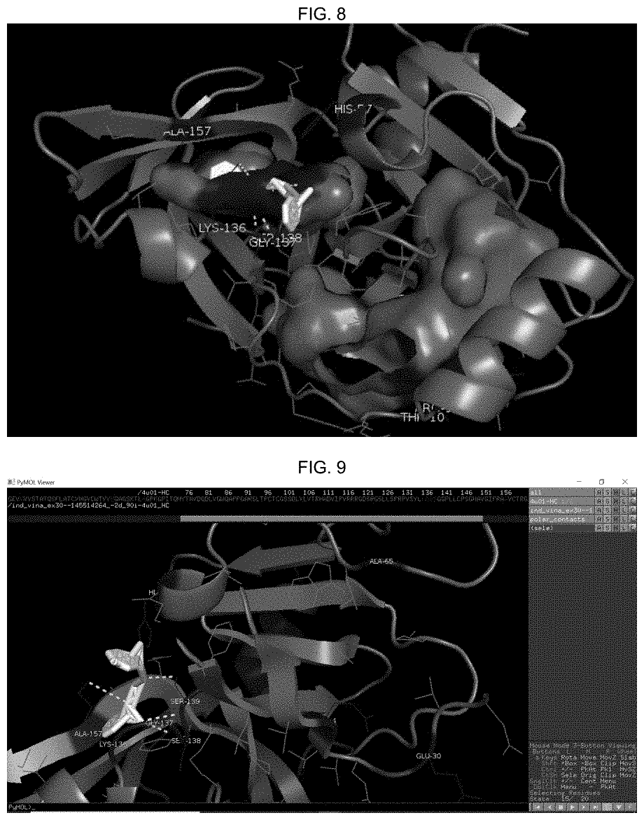

FIG. 8 illustrates 90I disposed within the binding pocket interacting with SER 136, GLY 137, SER 138, and SER 139.

FIG. 9 illustrates another pose of 90I interacting with SER 139, SER 138, LYS 135, and ALA157 with four H bonds.

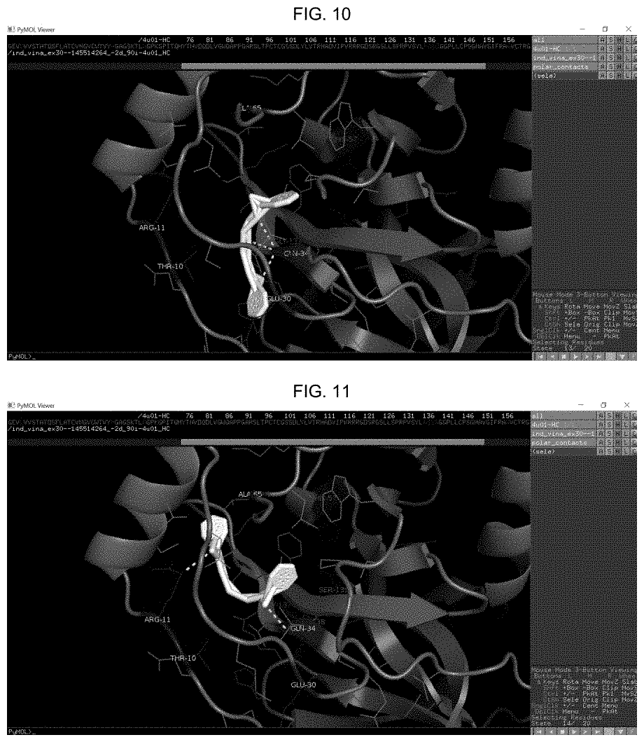

FIG. 10 illustrates 90I with additional H bonds interacting with residues.

Referring to FIG. 10, 90I may interact with three H bonds to make it stable. Moreover, the interaction with residues may be allosteric regions.

FIG. 11 illustrates 90I with additional H bond interactions with residues.

Referring to FIG. 11, the interaction with residues may also be allosteric regions.

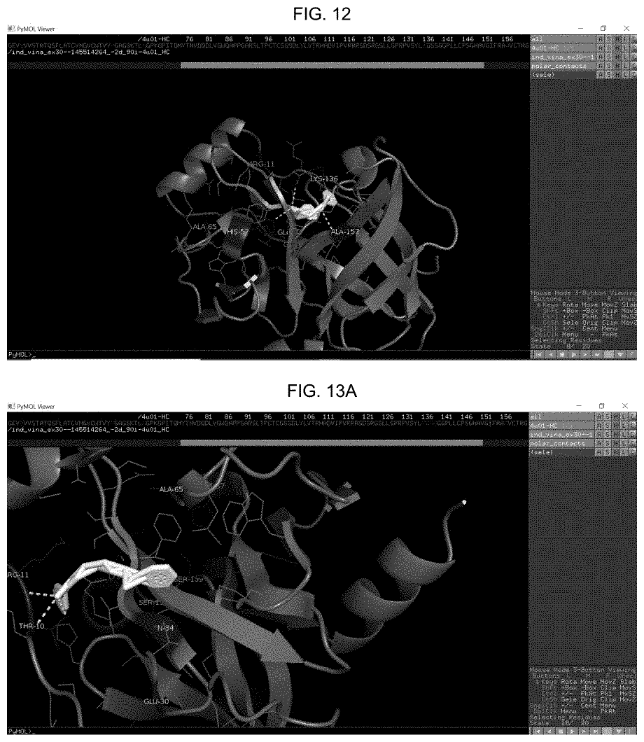

FIG. 12 illustrates 90I interacting with -LYS 136, ALA157, and HIS 57, including three H bonds.

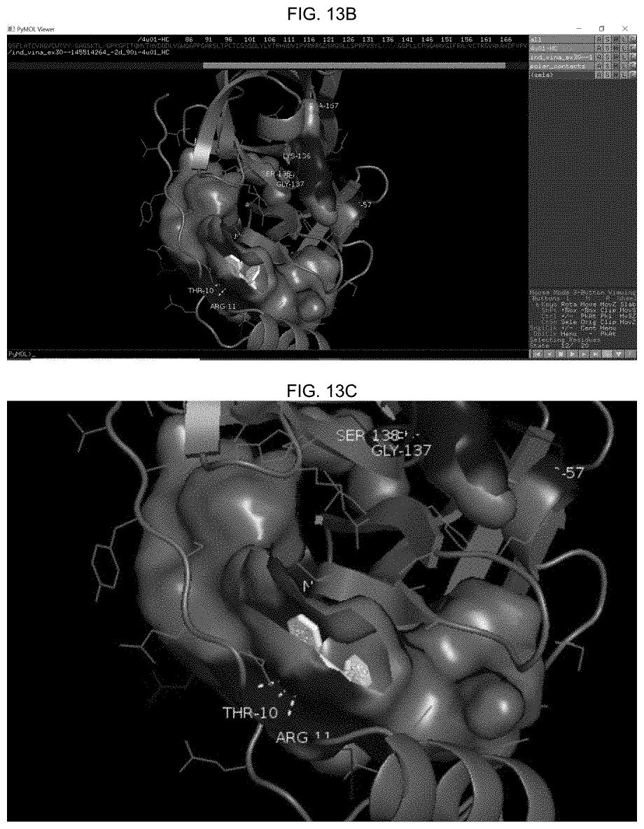

FIG. 13A illustrates 90I interacting with THR 10 and ARG 11.

FIG. 13B illustrates a pocket view of 90I interacting with THR 10 and ARG 11.

FIG. 13C illustrates another pocket view of 90I interacting with THR 10 and ARG 11.

FIG. 14 illustrates 90I interacting with GLN 34 and ARG 11 including two H bonds.

Referring to FIG. 14, the interaction with residues may also be allosteric regions.

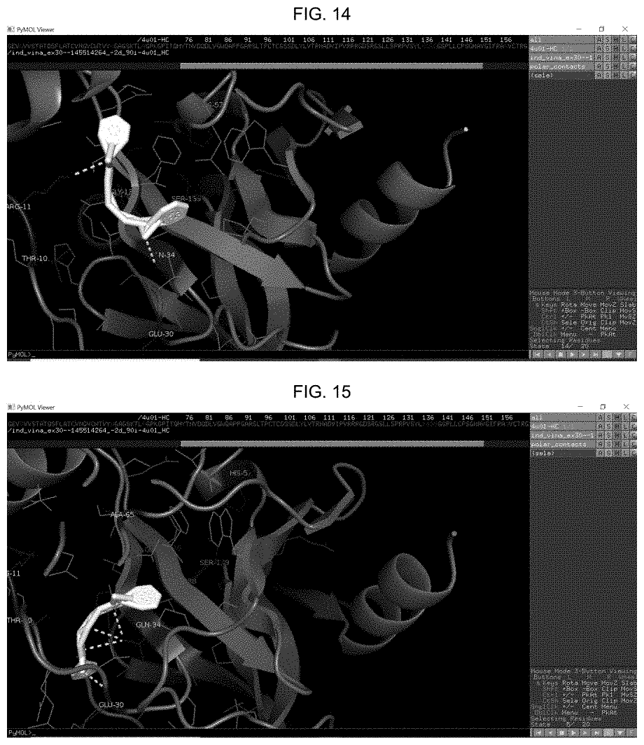

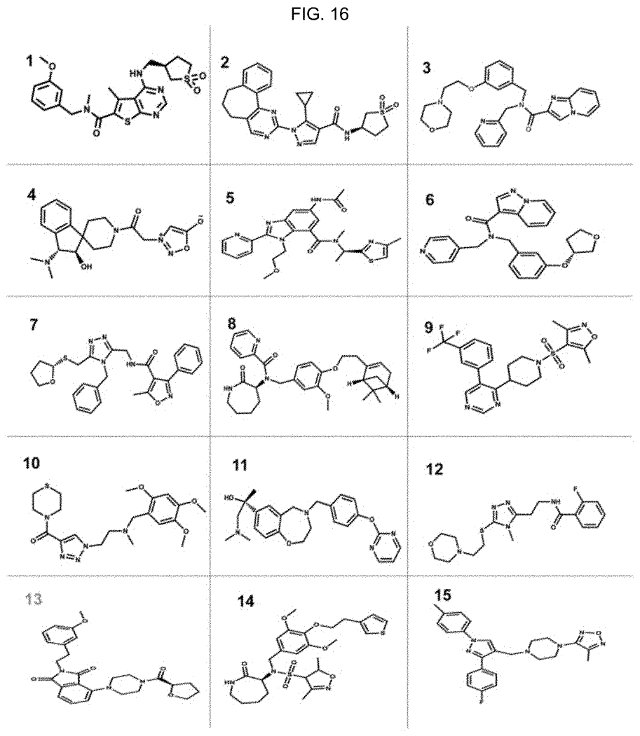

FIG. 15 illustrates 90I interacting with GLN 34 and GLU 30.

Referring to FIG. 15, the interaction with residues may also be allosteric regions.

In-Silico Helicase Study

The following is an excerpt from “In Silico Identification and Evaluation of Leads for the Simultaneous Inhibition of Protease and Helicase Activities of HCV NS3/4A Protease Using Complex Based Pharmacophore Mapping and Virtual Screening.” (See https://www.ncbi.nlm.nih.gov/pmc/articles/PMC3923879/).

In this study, the crystal structure of NS3/4A protease in complex with a macrocyclic inhibitor interacting with both protease and helicase (i.e. enzymes that bind and may even remodel nucleic acid or nucleic acid protein complexes) active sites residues.

The hydrogen bond acceptors features (Acc) were developed on the oxygen atoms of sulfonamide group and on the three carbonyl oxygen of the ligand owing to their binding interactions with important active site residues, Ser 139, Gly 137, Ala 157 and His 528. The hydrophobic (Hyd) feature locates the atom involved in interaction with His 57, the active site residue.

The resulted binding interactions between these 300 hits and protein were visually observed using LigPlot implemented in molecular operating environment (MOE) and those molecules which revealed significant interactions with most of the important binding pocket residues (His 57, Lys 136, Ser 139, Gly 137, Arg 155, Ala 157, Ala 156 of protease site and Met 485, Glu526, His 528 of helicase site) of HCV NS3/4A protease were selected as promising hits.

As discussed above, affinity is calculated in kcal/mol. The lesser the better. As shown below, there are other drug like molecules that are not IDX320, but other drugs to illustrate affinity of drugs and the scale of effectiveness versus affinity.

90I may have affinity of −7 kcal/mol at 0 A distance RMSD value (See 90I log files above). These molecules can also be used to compare with 90I for additional confirmation in addition to the in-vitro that was done using IDX320.

The in-silico experiment identifies different affinity scores generated. However, only the binding affinity is provided for sake of comparison with 90I that will be generated.

FIG. 16 illustrates 2D structures of lead compounds to acts as novel, potent, and structurally diverse inhibitors of HCV NS3/4A protease. (See https://www.ncbi.nlm.nih.gov/pmc/articles/PMC3923879/).

TABLE 2

| | | Docking | Binding | |
|---|---|---|---|---|
| Com- | | Score | affinityKcal/ | |
| pound | ChemBridgeID | (S) | mol | Drug like properties |
| 1 | 74212070 | −12.1129 | −6.40 | MW. 474.61 g/mol, |
| | | | | LogP. 3.39, LogS. |
| | | | | −5.37, Don. 1, Acc. |
| | | | | 6 |
| 2 | 13203524 | −11.4164 | −6.17 | MW. 463.56 g/mol, |
| | | | | LogP. 2.61, LogS. |
| | | | | −5.71, Don. 1, Acc. |
| | | | | 6 |
| 3 | 20259391 | −11.1719 | −6.71 | MW. 471.56 g/mol, |
| | | | | LogP. 3.86, LogS. |
| | | | | −3.48, Don. 0, Acc. |
| | | | | 6 |
| 4 | 27798935 | −11.0684 | −5.92 | MW. 372.42 g/mol, |
| | | | | LogP. 1.01, LogS. |
| | | | | −2.12, Don. 1, Acc. |
| | | | | 5 |
| 5 | 92175699 | −11.0708 | −7.38 | MW. 492.60 g/mol, |
| | | | | LogP. 4.66, LogS. |
| | | | | −4.60, Don. 1. Acc. |
| | | | | 6 |
| 6 | 63465583 | −10.9940 | −5.99 | MW. 429.48 g/mol, |
| | | | | LogP. 4.01, LogS. |
| | | | | −3.47, Don. 0, Acc. |
| | | | | 6 |
| 7 | 60321457 | −10.9496 | −7.87 | MW. 489.60 g/mol, |
| | | | | LogP. 5.02, LogS. |
| | | | | −7.06, Don. 1, Acc. |
| | | | | 5 |
| 8 | 93854211 | −10.8731 | −7.98 | MW. 517.67 g/mol, |
| | | | | LogP. 5.43, LogS. |
| | | | | −6.53, Don. 1, Acc. |
| | | | | 5 |
| 9 | 34215248 | −10.8285 | −6.46 | MW. 466.48 g/mol, |
| | | | | LogP. 4.65, LogS. |
| | | | | −5.37, Don. 0, Acc. |
| | | | | 5 |
| 10 | 97464457 | −10.6463 | −7.37 | MW. 435.55 g/mol, |
| | | | | LogP. 2.16, LogS. |
| | | | | −2.39, Don. 0, Acc. |
| | | | | 7 |
| 11 | 10355774 | −10.6286 | −7.54 | MW. 434.54 g/mol, |
| | | | | LogP. 4.28, LogS. |
| | | | | −4.51, Don. 1, Acc. |
| | | | | 6 |
| 12 | 45481066 | −10.6055 | −7.52 | MW. 393.49 g/mol, |
| | | | | LogP. 1.71, LogS. |
| | | | | −3.79, Don. 1, Acc. |
| | | | | 5 |
| 13 | 51314220 | −10.6047 | −7.70 | MW. 463.53 g/mol, |
| | | | | LogP. 2.36, LogS. |
| | | | | −4.65, Don. 0, Acc. |
| | | | | 5 |
| 14 | 35611883 | -10.5838 | −7.25 | MW. 563.70 g/mol, |
| | | | | LogP. 4.12, LogS. |
| | | | | −4.90, Don. 1, Acc. |
| | | | | 7 |
| 15 | 37363620 | −10.4503 | −6.48 | MW. 432.50 g/mol, |
| | | | | LogP. 4.27, LogS. |
| | | | | −5.71, Don. 0, Acc. |
| | | | | 4 |
| 16 | Reference | −12.789 | −11.25 | MW. 865.96 g/mol, |
| | | | | LogP. 3.17, LogS. |
| | | | | −8.61, Don. 4, |
| | | | | Acc.8 |

Referring to Table 2, the data included shows ChemBridge database ID, Docking Scores, binding energies, binding affinities and drug like properties of hit compounds on HCV protease.

Identified below is another Protease example with HIS 528 (i.e. a helicase binding residue).

Another x-ray image of protease from PDB. This time another x-ray ligand is bound to a helicase residue, whereas previously IDX320 was used on a protease. As such, another x-ray was performed on a ligand on helicase interacting with HIS 528, an important residue.

A PDB file was generated from an x-ray of actual crystalized protein. This PDB file x-ray image also contains a docked ligand on a same protein. This x-ray image is reliable and can be used as a control or reference to compare it with 90I. PyMOL was used to obtain information by extracting their binding sites. Subtrait binding domain or allosteric residues that will affect the helicase will be identified since this is a real x-xray image.

Furthermore, the x-ray image with the ligand in it to additionally identify where the ligand should dock to inhibit and determined whether 90I also will inhibit the residues, THR 160, HIS 528, CYS 591, GLN 526, GLY 137, and SR 139 according to the real x-ray image H bond from PDB.

The following is an excerpt from “Understanding the Structural and Energetic Basis of Inhibitor and Substrate Bound to the Full-Length NS3/4A: Insights From Molecular Dynamics Simulation, Binding Free Energy Calculation and Network Analysis.” (See https://pubmed.ncbi.nlm.nih.gov/22833015/).

The NS3/4A substrate and inhibitor envelopes reveals the areas where the consensus inhibitor volume extended beyond the substrate envelope correspond to drug resistance mutations including Arg155, Ala156 and Asp168 at the protease active site as well as the two conserved helicase residues Gln526 and His528 that strongly interact with the inhibitors. Thus, the findings of this study will be very useful for understanding the interaction mechanism between the inhibitor (substrate) and NS3/4A and also for the rational design and development of new potent molecules targeting the full-length NS3/4A.

Full-length HCV NS3-4A protease-helicase in complex with a macrocyclic protease inhibitor. (See https://www.rcsb.org/structure/4a92). 4A92.

Method: X-RAY DIFFRACTION.

A Macrocyclic HCV NS3/4A Protease Inhibitor Interacts with Protease and Helicase Residues in the Complex with its Full-Length Target. (See https://pubmed.ncbi.nlm.nih.gov/22160684/).

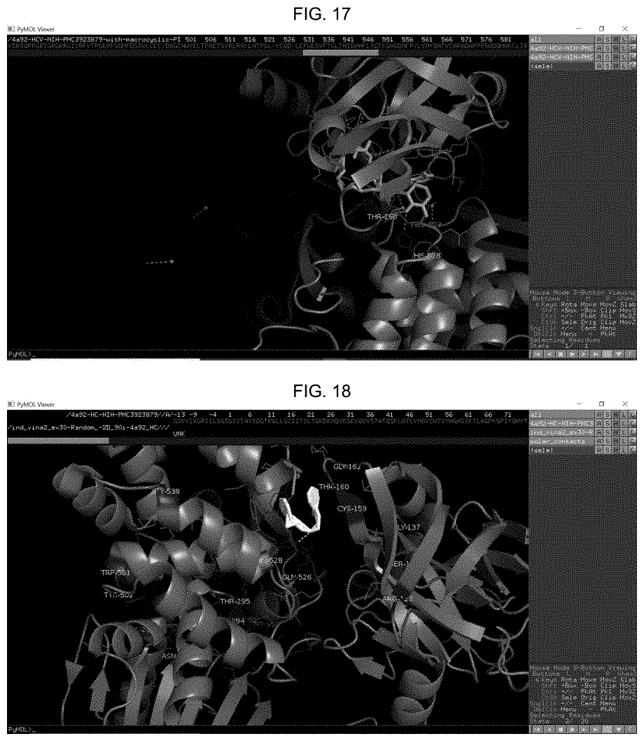

FIG. 17 illustrates an x-ray image of 4a92 PDB with PI from PDB including a ligand in orange on protein with H bond in yellow and residues THR 160 and HIS 528 in blue.

Referring to FIG. 17, 90I interacts with both residues.

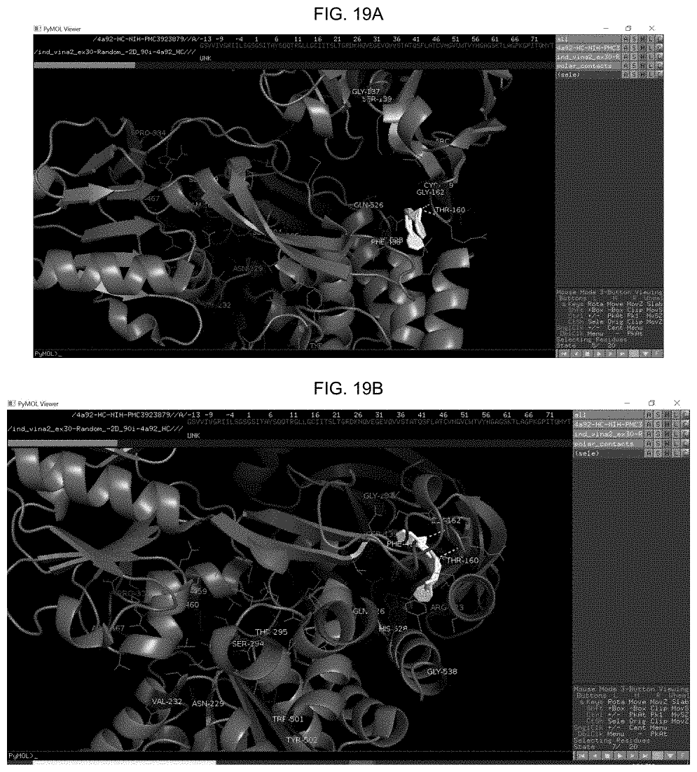

FIG. 18 illustrates a second pose of 90I interacting with HIS 528.

Referring to FIG. 18, 90I may have 0.0164 Argon distance RMSD and −7.3 kcal/mol (See table 3 for log files). As such, the second pose of 90I including data from the above log files is an absolutely good hit.

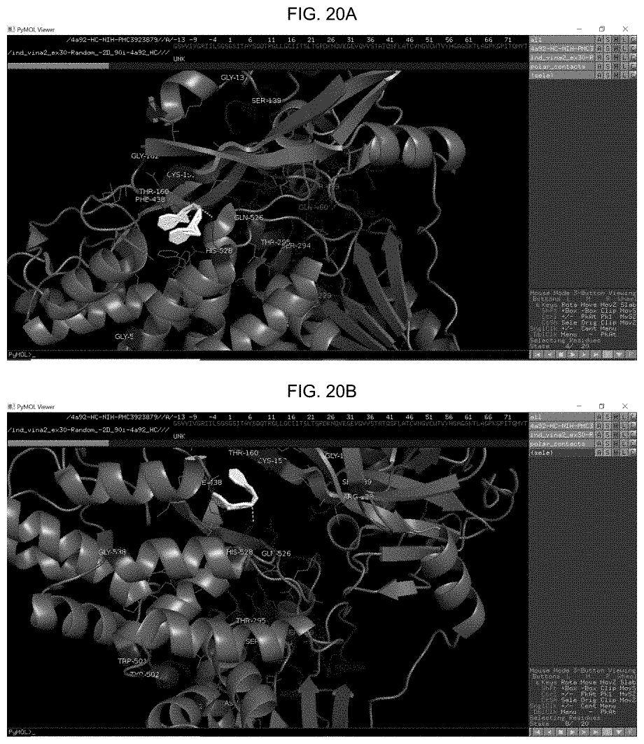

FIG. 19A illustrates 90I interacting with residues THR 160 and GLY 162.

Referring to FIG. 19A, 90I is shown similar to the x-ray ligand interaction in FIG. 17.

FIG. 19B illustrates another pose of 90I interacting with residues THR 160 and GLY 162.

Referring to FIG. 19B, 90I is shown similar to the x-ray ligand interaction in FIG. 17.

FIG. 20A illustrates another pose of 90I interacting with residue HIS 528 with an H bond.

FIG. 20B illustrates another pose of 90I interacting with residue HIS 528.

Referring to FIG. 20B, 90I may have many interactions with HIS 528 as shown before.

FIG. 20C illustrates another pose of 90I interacting with residue HIS 528.

Referring to FIG. 20C, 90I may have many interactions with HIS 528 as shown before.

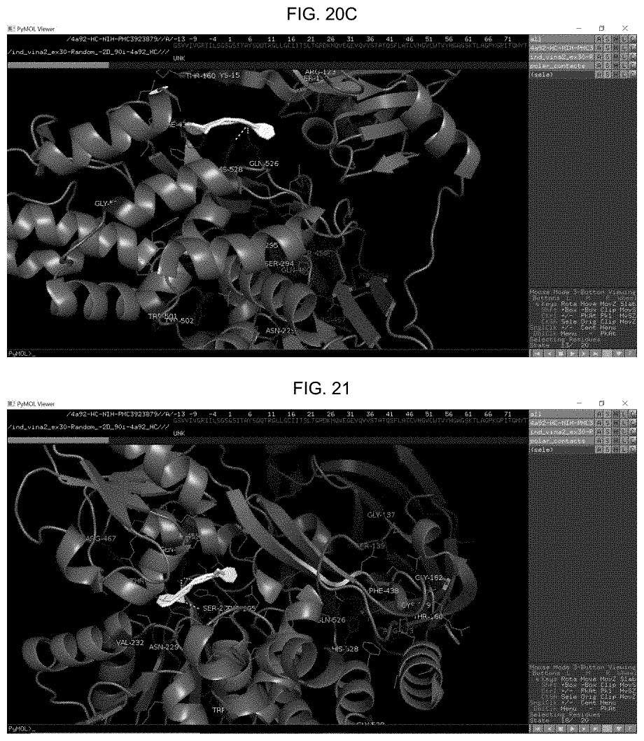

FIG. 21 illustrates 90I Interacting with residue SER including three H bonds.

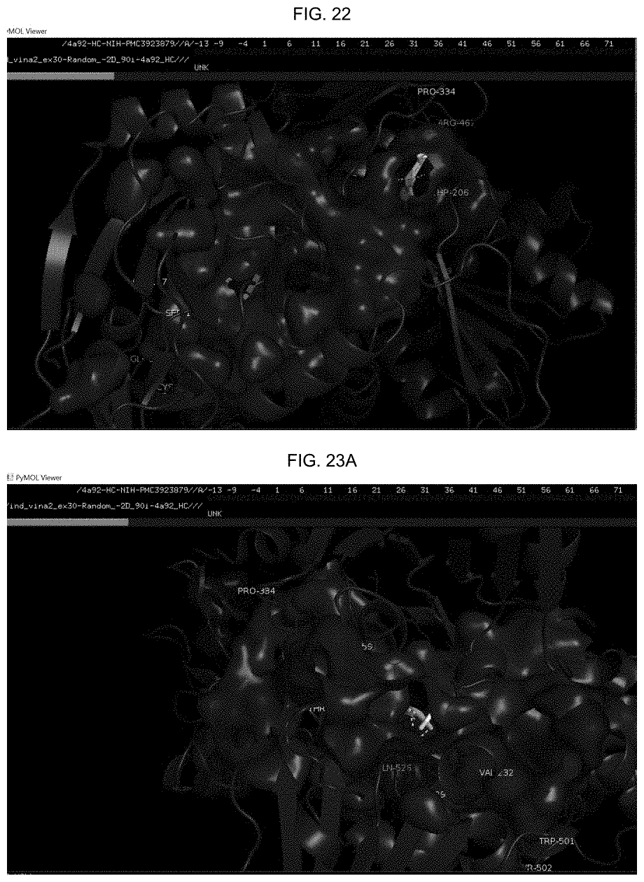

FIG. 22 illustrates 90i disposed within a binding pocket.

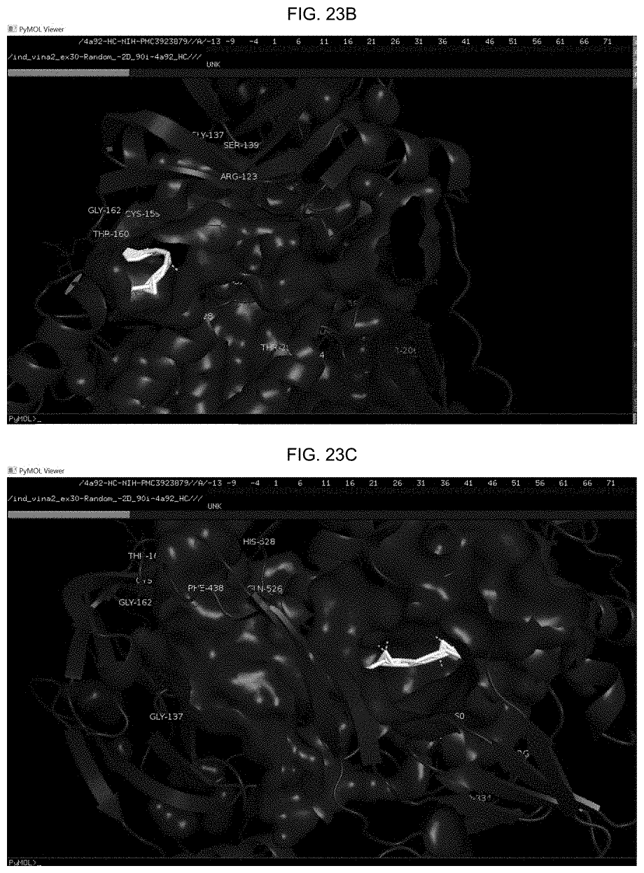

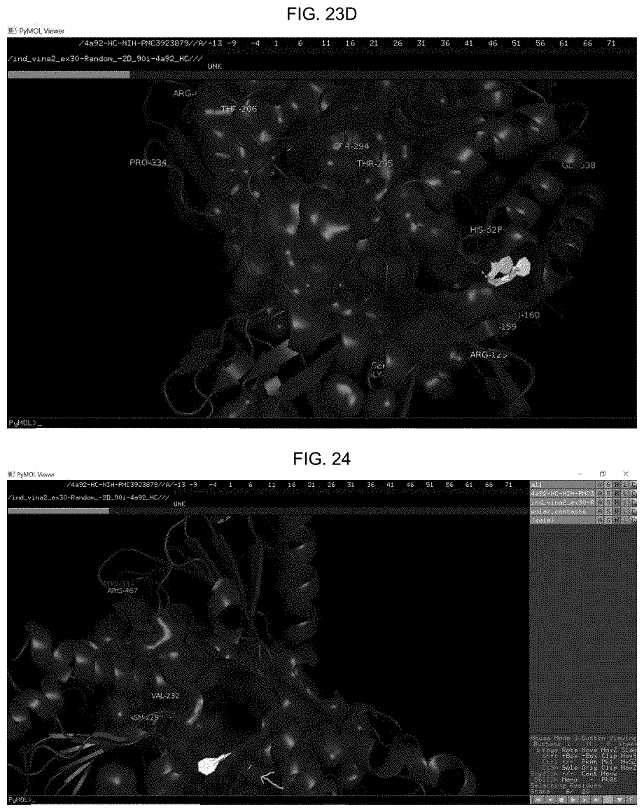

FIG. 23A illustrates 90I disposed within a binding pocket while interacting with residue HIS 528.

FIG. 23B illustrates another view of 90I disposed within the binding pocket.

FIG. 23C illustrates a different view of 90I disposed within the binding pocket.

FIG. 23D illustrates 90I disposed within the binding pocket interacting with HIS 528.

FIG. 24 illustrates 90I disposed within a binding pocket interacting with at least one residue and creating an H bond thereto.

Referring to FIG. 24, 90I may create a stable connection to the H bond.

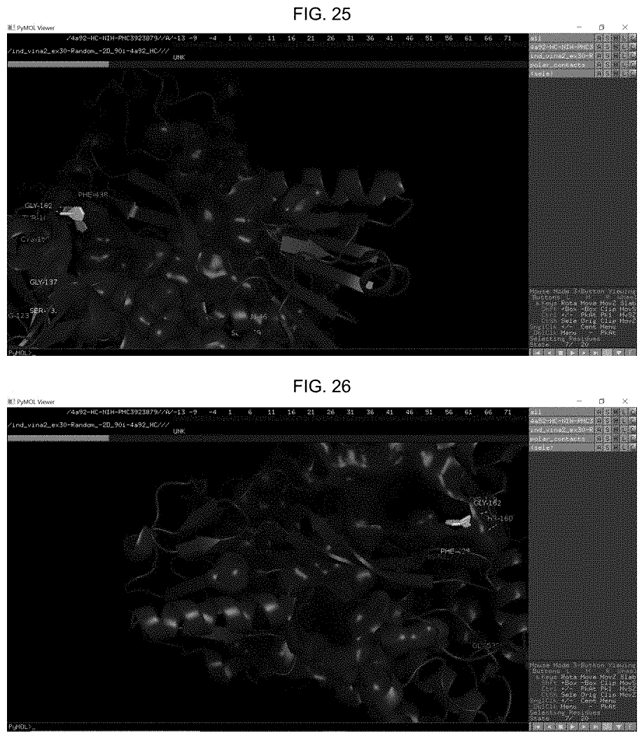

FIG. 25 illustrates 90I disposed within a binding pocket interacting residue GLY 162 including two H bonds.

FIG. 26 illustrates 90I disposed within a binding pocket interacting with residues, GLY 162 and THR 160.

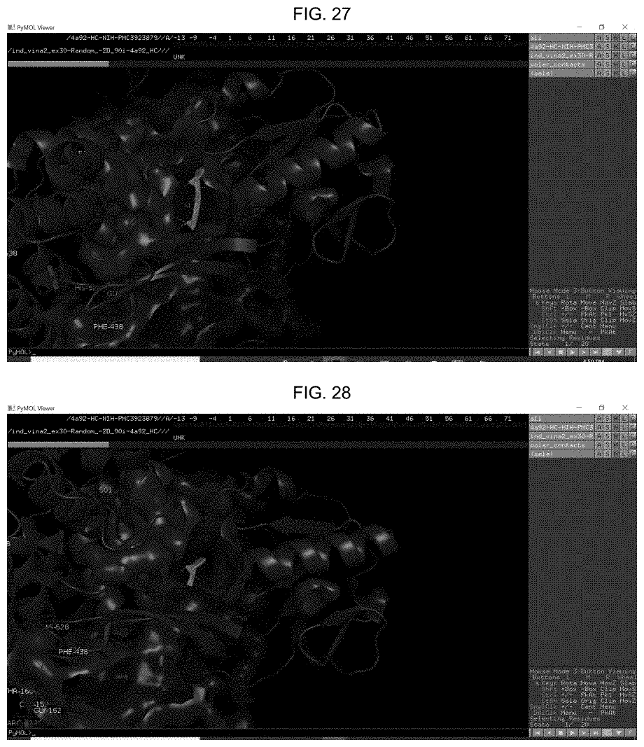

FIG. 27 illustrates 90I disposed within a binding pocket with four H bonds.

FIG. 28 illustrates 90I disposed deep within a binding pocket.

Referring to FIG. 28, 90I may be affecting the protein connected thereto.

The excerpt identified above “Understanding the Structural and Energetic Basis of Inhibitor and Substrate Bound to the Full-Length NS3/4A: Insights From Molecular Dynamics Simulation, Binding Free Energy Calculation and Network Analysis” disclosed another study regarding HIS 528, GLN 526, ASP 168, and ASP 156. (See https://pubmed.ncbi.nlm.nih.gov/22833015/).

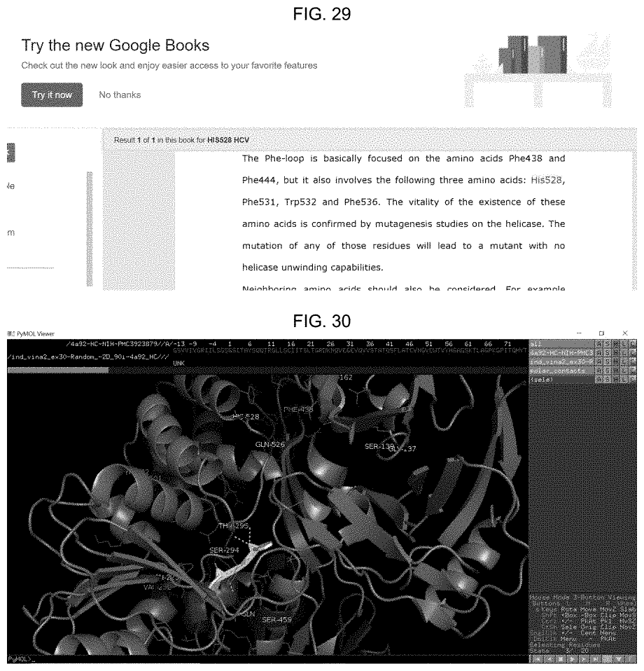

FIG. 29 illustrates a book excerpt regarding residues.

The following is an excerpt from “Simultaneously Targeting the NS3 Protease And Helicase Activities For More Effective Hepatitis C Virus Therapy.” (See https://www.ncbi.nlm.nih.gov/pmc/articles/PMC4546510/).

This study examines the specificity and mechanism of action of a recently reported hepatitis C virus (HCV) non-structural protein 3 (NS3) helicase-protease inhibitor (HPI), and the interaction of HPI with the NS3 protease inhibitors telaprevir, boceprevir, danoprevir, and grazoprevir.

HCV genotype 1 NS4A/NS3 proteins harboring amino acid near the binding site of peptidomimetic protease inhibitors residues Val524, Gln526, His528 and F438.

Also, Thr295 contacts the other end of helicase-protease inhibitor (HPI) and Thr435 contacts the center of HPI.

FIG. 30 illustrates 90I interacting with residues THR 295, SER 294, SER 459, and GLN 460 including four H bonds.

Referring to FIG. 30, 90I shows −7.3 kcal/mol even if the distance is far. Moreover, an H bond is still present.

The following is an excerpt from “A macrocyclic HCV NS3/4A protease inhibitor interacts with protease and helicase residues in the complex with its full-length target.” (See https://www.ncbi.nlm.nih.gov/pmc/articles/PMC3248494/).

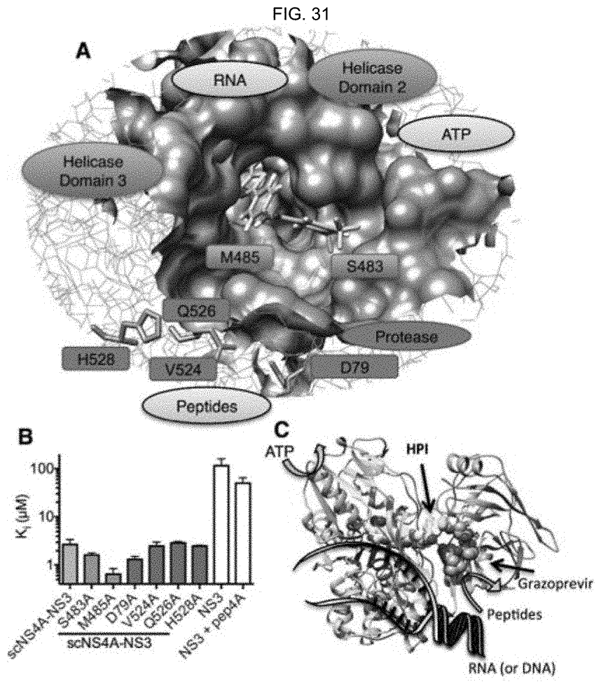

FIG. 31 illustrates a possible HPI binding site on NS3.

Interactions with Helicase Residues.

The P4-capping and P2 moieties of inhibitor 1 are exposed toward the helicase interface and interact both with protease and helicase residues. The inhibitor buries 170 Å² of accessible surface area (ASA) of helicase residues involving in particular residues Met485 and the segment Val524 to His528. Upon inhibitor binding there are two significant changes in the crystal involving helicase residues in the active site. Residues beyond Ala625 are disordered, i.e., the helicase C terminus is not represented by electron density. The nonprime portions of the inhibitor take on the position occupied by helicase residues Glu628, Val629, Val630, and Thr631 in the apo structure.

The following is another excerpt from “In Silico Identification and Evaluation of Leads for the Simultaneous Inhibition of Protease and Helicase Activities of HCV NS3/4A Protease Using Complex Based Pharmacophore Mapping and Virtual Screening.” (See https://www.ncbi.nlm.nih.gov/pmc/articles/PMC3923879/).

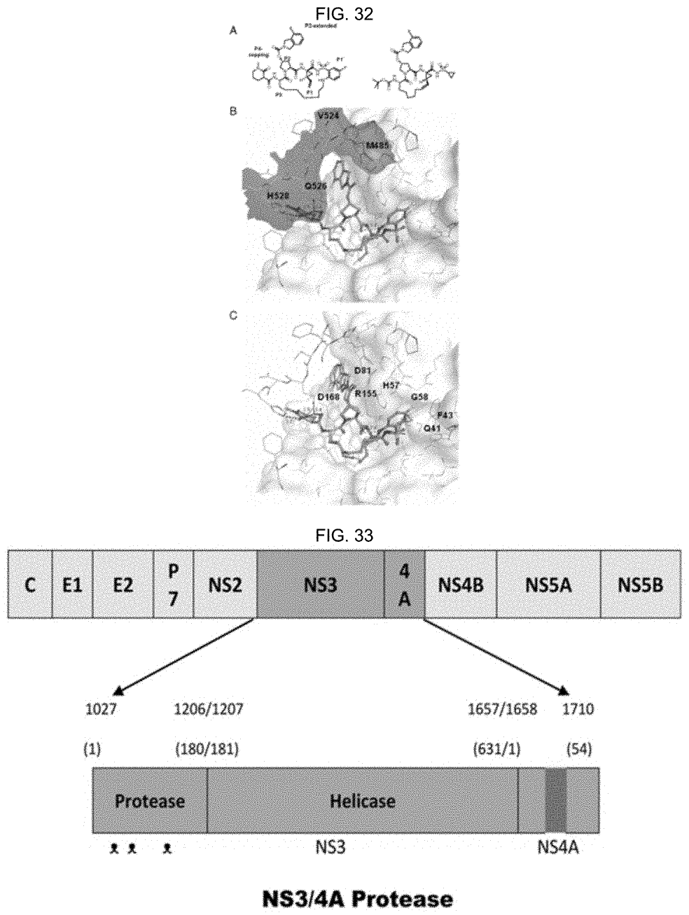

FIG. 32 illustrates an inhibitor 1 bound to a HCV NS4/4A.

Materials and Methods

Generation and Validation of Complex-Based Pharmacophore Model

Pharmacophore is an ensemble of steric and electronic features that is necessary to ensure the optimal supramolecular interactions with a specific biological target and to block its biological response. The complex-based pharmacophore technique can be used to advance the drug development process if the 3D structure of the target protein is available. In this study the crystal structure of NS3/4A protease in complex with a macrocyclic inhibitor interacting with both protease and helicase active sites residues (4a92) was used for the generation of complex-based pharmacophore model.

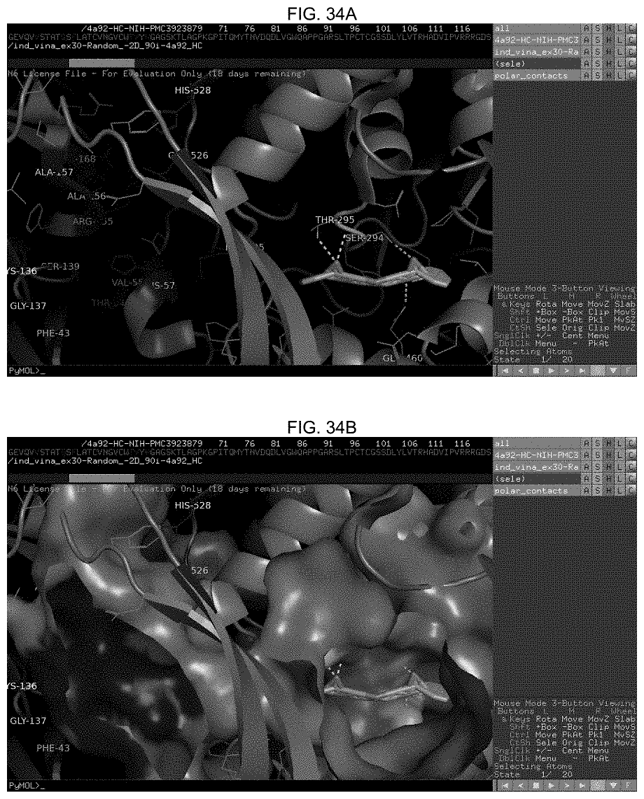

FIG. 33 illustrates a schematic representation of the HCV NS3/4A protease. (See https://www.ncbi.nlm.nih.gov/pmc/articles/PMC3923879/).

The amino acid position for the domain and sub-domain is indicated as a number either starting from the 1st amino acid of the entire polyprotein (the number at the top) or starting from the 1st amino acid of the NS3 or NS4A (the number at the bottom). On the NS3/4A protease, the catalytic triad, namely His-1083, Asp-1107 and Ser-1165 of the polyprotein (or His-57, Asp-81 and Ser-139 of the NS3), is indicated as “i”. The reddish box in the NS4A indicates the 14-amino acid central hydrophobic region of NS4A (amino acids 1678-1691 of the polyprotein or amino acids 21-34 of the NS4A), which has been shown to be sufficient for activation of the NS3 protease activity.

The active site configuration of NS3 protease comprises the residues His-57 (His-1083), Asp-81 (Asp-1107), and Ser-139 (Ser-1165). NS3 protease requires the vital 14-monomer hydrophobic peptide NS4A for its activation

The hydrogen bond acceptors features (Acc) were developed on the oxygen atoms of sulfonamide group and on the three carbonyl oxygen of the ligand owing to their binding interactions with important active site residues, Ser 139, Gly 137, Ala 157 and His 528.

Binding Interactions of Finally Selected Compounds (See https://www.ncbi.nlm.nih.gov/pmc/articles/PMC3923879/).

It was observed in the docking studies that all finally selected hits showed significant binding interactions with the important residues of protease as well as helicase site of the target protein. For example, compound 1, for which the strong binding affinity (−6.40 Kcal/Mol), lower binding energy (−23.04 Kcal/Mol) and good docking score (−12.1129) was observed, showed the binding interaction with the protease and helicase binding site residues. From the top-ranked docked conformation, it was also observed that a phenyl ring, thienopyrimidine moiety and an adjacent carbonyl oxygen of the compound 1 established interactions with the residues of helicase binding site of the target protein whereas a terminal methoxy group and two oxygen atoms of thiophene interact with the protease binding site residues. The helicase binding site residues, His 528 made an arene-hydrogen interaction with the phenyl ring and Gln 526 made two polar interactions with the carbonyl oxygen and thienopyrimidine moiety. From the protease site, the residues, His 57, Ser 139, and Gly 137 are involved in hydrogen bonding with oxygen atoms of thiophenering and Cys 159 with the methoxy group of compound.

According to the docking score, comparatively low ranked hits also showed significant interactions with important residues of protease and helicase sites. For example, in compound 13 it was observed that isoindolinedione ring of compound form arene-hydrogen bonds to His57 and Ala 156 of protease site residues. Beside these residues of protease site Ala 157 also showed hydrophobic interaction to a methylene group of compound. Similarly, helicase site residues, Met 485, Gln 526 and His 528 were observed to be involved in intermolecular interactions with various groups of compound 13. Met 485 and Gln 526 are involved in hydrogen bonding with furan group and carbonyl oxygen of isoindolinedione ring respectively. Gln 526 also showed a second polar hydrogen acceptor bonding to hydrogen of adjacent methylene group. An arene-hydrogen bonding was also observed between His 528 and phenyl ring of compound 13. The pharmacophore mapping of compound 13 is shown in a 54-residue long important cofactor for NS3 proteolytic activity. For activation of the NS3 protease domain, only residues 21 to 34 of NS4A are required.

The resulted binding interactions between these 300 hits and protein were visually observed using LigPlot implemented in MOE and those molecules which revealed significant interactions with most of the important binding pocket residues (His 57, Lys 136, Ser 139, Gly 137, Arg 155, Ala 157, Ala 156_of protease site and Met 485, Glu526, His 528 of helicase site) of HCV NS3/4A protease were selected as promising hits. Among these 300 compounds, 52 showed crucial interactions with the important residues of target protein. These 52 compounds were further subjected to Binding energy and Binding affinity calculation.

Active site residues ARG 155, ALA 156 and ASP 168 are prone to multi-drug resistance.

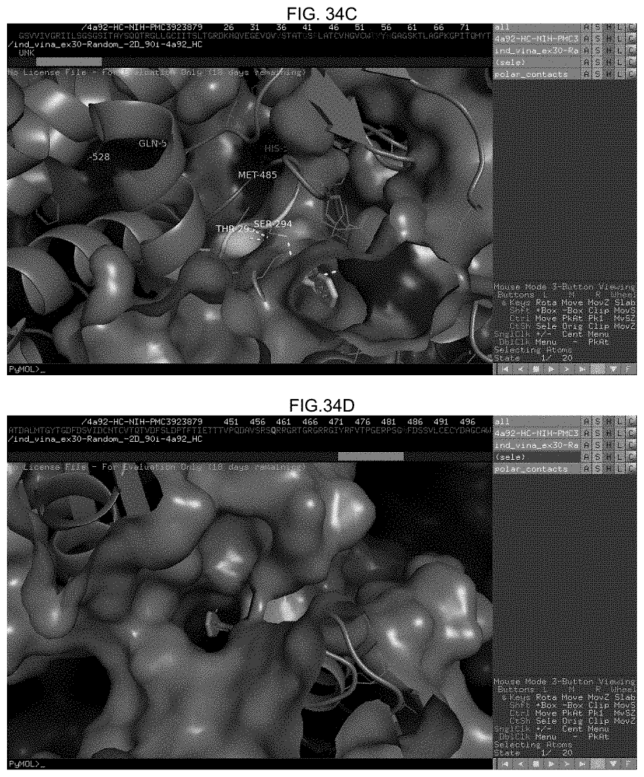

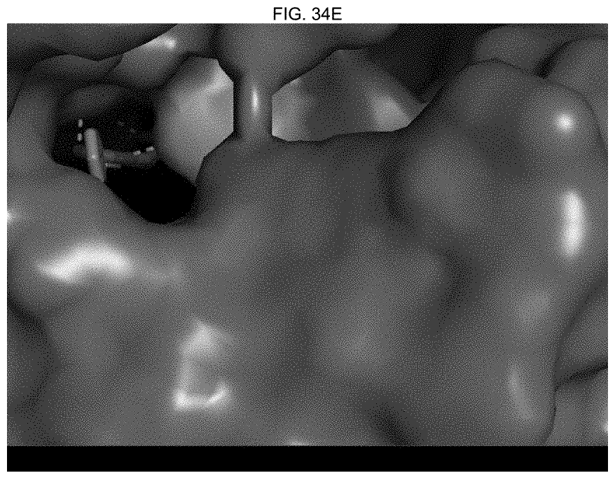

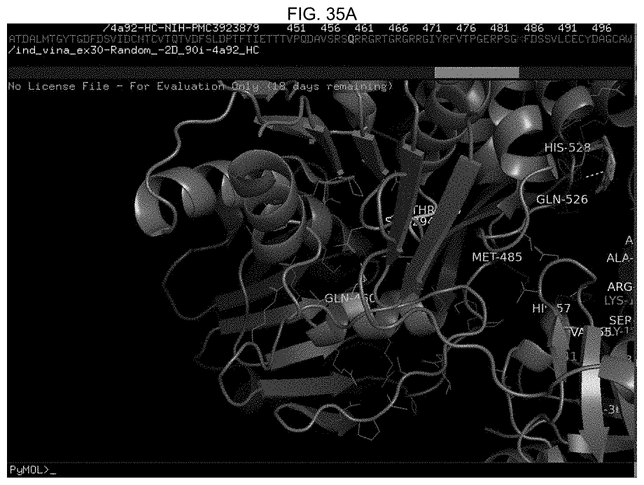

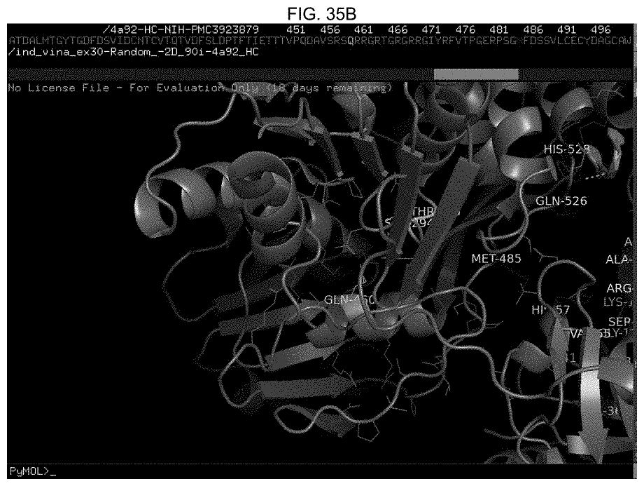

FIG. 34A illustrates a first pose of 90I disposed within a binding pocket with four H bonds.

Referring to FIG. 34A, 90I shows strong H bonds.

FIG. 34B illustrates another view of the first pose of 90I disposed within the binding pocket with four H bonds.

FIG. 34C illustrates a 12 A zoomed in first surface view of the first pose of 90I disposed within the binding pocket.

FIG. 34D illustrates another zoomed in full surface view of the first pose of 90I disposed within the binding pocket.

FIG. 34E illustrates another zoomed in view of 90I disposed within the binding pocket interacting with an H bond.

Referring to FIG. 34E, 90I shows a strong fit inside the binding pocket.

FIG. 35A illustrates a different pose of 90I interacting with a residue HIS 528.

FIG. 35B illustrates 90I interacting with the residue HIS 528.

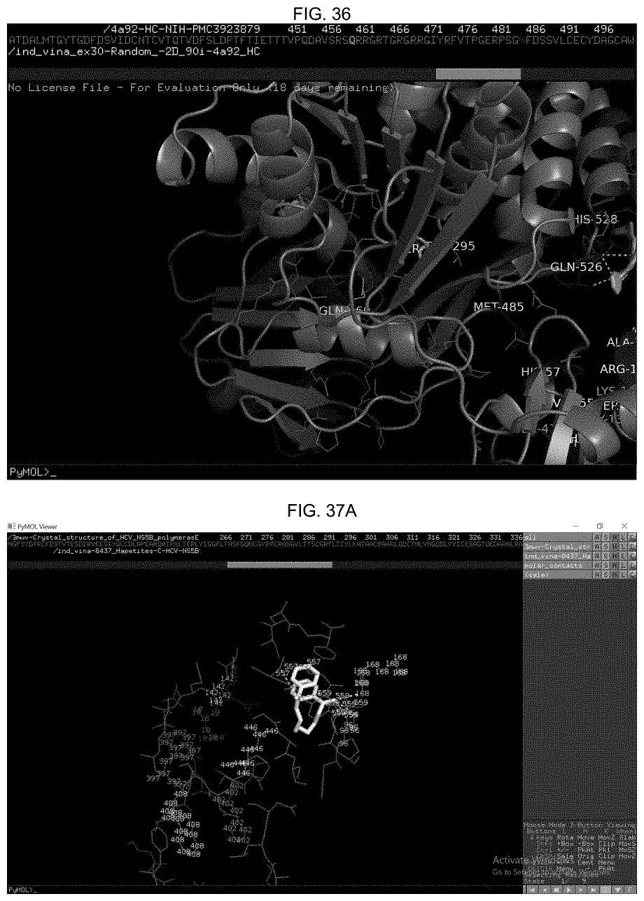

FIG. 36 illustrates 90I interacting with residues HIS 528 and GLN 526 including three H bonds.

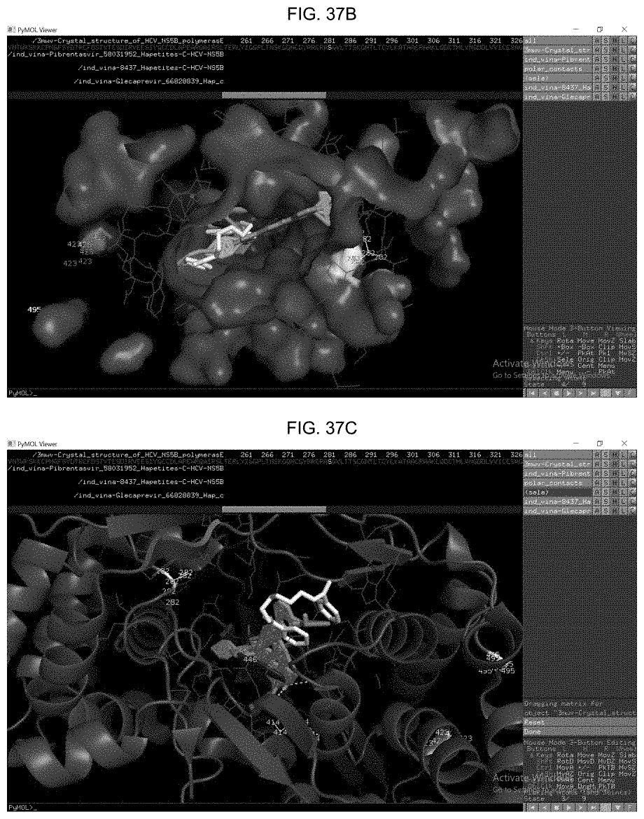

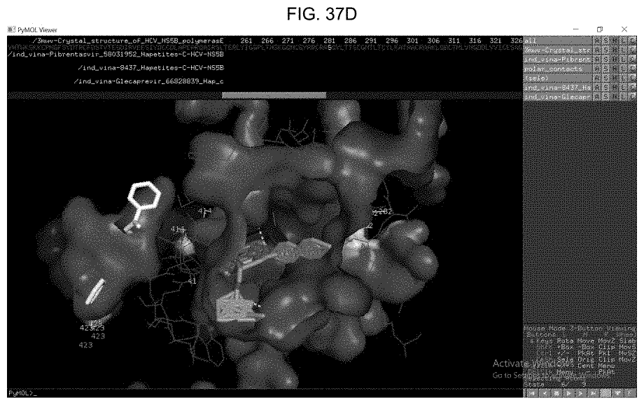

FIG. 37A illustrates 90I interacting with polymerase inhibitors, glecaprevir and pibrentasvir, as controls.

FIG. 37B illustrates 90I in white disposed on polymerase and a plurality of binding pockets interacting with polymerase inhibitors, glecaprevir and pibrentasvir.

Referring to FIG. 37B, 90I demonstrates similar behavior as the polymerase inhibitors, but is less toxic and more, as described above.

FIG. 37C illustrates 90I in white disposed on polymerase and the plurality of binding pockets interacting with polymerase inhibitors, glecaprevir and pibrentasvir.

Referring to FIG. 37C, 90I demonstrates similar behavior as the polymerase inhibitors, but is less toxic and more, as described above

FIG. 37D illustrates 90I in white disposed on polymerase and within at least one binding pocket interacting with polymerase inhibitors, glecaprevir and pibrentasvir.

Referring to FIG. 37D, although, 90I is able to enter the at least one binding pocket, the two drugs, glecaprevir and pibrentasvir, do not go in. Moreover, in-silico analysis indicates 90I may inhibit polymerase as well.

Also, based on the following study on other drugs, 90I may behave similarly, if not better.

The following is an excerpt from “NS5B RNA Dependent RNA Polymerase Inhibitors: The Promising Approach to Treat Hepatitis C Virus Infections.” (See https://pubmed.ncbi.nlm.nih.gov/20858218/).

Hepatitis C virus (HCV), a causative agent for non-A and non-B hepatitis, has infected approximately 3% of world's population. The current treatment option of ribavirin in combination with pegylated interferon possesses lower sustained virological response rates, and has serious disadvantages. Unfortunately, no prophylactic vaccine has been approved yet. Therefore, there is an unmet clinical need for more effective and safe anti-HCV drugs. HCV NS5B RNA dependent RNA polymerase is currently pursued as the most popular target to develop safe anti-HCV agents, as it is not expressed in uninfected cells. More than 25 pharmaceutical companies and some research groups have developed h50 structurally diverse scaffolds to inhibit NS5B. Here we provide comprehensive account of the drug development process of these scaffolds. NS5B polymerase inhibitors have been broadly classified in nucleoside and non nucleoside inhibitors and are sub classified according to their mechanism of action and structural diversities. With some additional considerations about the inhibitor bound NS5B enzyme X-ray crystal structure information and pharmacological aspects of the inhibitors, this review summarizes the lead identification, structure activity relationship (SAR) studies leading to the most potent NS5B inhibitors with subgenomic replicon activity.

As shown above, 90I may be an all natural, low cost, and non-toxic treatment in targeting one of the most highly infectious diseases which has crippled and burdened governments worldwide, especially third world countries. From the standpoint of the customer, most infectious cases affect individuals who cannot afford the current available treatments. As such, investing in 90I as an alternative will alleviate the financial burden of the patients and decrease the need for treatment of side effects caused by the current available anti-HCV drugs on the market.

Antiviral Drug Assay

Standard anti-viral drug evaluation methods will be used in this study. 90I will be evaluated against all four HCV drugs. Ribavirin, an FDA approved commercially available anti-HCV drug, will used as control drug for the functionality of the study. Toxicity control will be included since toxicity is one of the big issues in HCV therapy. Virus and cell control will be included for the functionality of assay.

In addition to the above evaluation, a combination of drugs including 90I and ribavirin will be used at low concentration against HCV to determine synergy or antagonism with commercial HCV drugs.

This study will further evaluate the mode of actions of 90I against HCV, to determine whether it is early, late, late-late, or a combination thereof.

| | | | |
|---|---|---|---|
| Assay Type: | Mode of Action | Time | Segments the Life |
| Test drug: | 90I | Course: | Cycle of HIV-1 |
| Control | ribavirin and albinterferon | | sequential stages |
| Drugs | | Cell: | Huh 7 |
| Experimental | Time Course Drug | Virus: | HCV G1 |
| | addition at a single | MOI: | >1.5 × 10\hat{ }6 RNA |
| | Concentration of 1 uM | | Copy at 0 hr. |
| Set-up: | each drug will be added | | |
| | at 0 hr of infection | | |
| | and 4 hr, 24 hr. 48 hr, | | |
| | 72 hr and at 96 hrs of | | |
| | post infection. | | |

An Amplicor system will be used to automate amplification and detection of target nucleic acids, making diagnostic polymerase chain reaction (PCR) routine for infectious diseases. Amplicor will be applied to HCV PCR, RNA copies, albinterferon, enzyme-linked immunosorbent assay (ELISA), and MTS for end point determination.

Also, EC50 and TI will be the benchmark of the drug evaluation.

### REFERENCES

The following reference(s) may provide exemplary procedural and/or other details supplementary to those set forth herein, and are specifically incorporated herein by reference.
1. Zeuzem S. et al Pegiinterferon alfa-2a in patients with chronic hepatitis C N. Eng. J Med 2000: 343: 1666-1672.
2. Shah P J. Tolbutamide and fatal water intoxication. Br J Psychiatry 1991 158: 719-720.
3. Rustigi V K. Albinterferon alfa 2b, a novel fusion protein of a human. albumin and human interferon alfa 2b, for chronic hepatitis C. Curr Med Res Opin. 2009:25: 991-1002.
4. Cohen D M. Antiviral drug resistance. Ann NY Acad Sci: 1990: 616: 224-237.
5. Tilak J C et al. Antioxident properties of Plumbango zeylanica, an Indian medicinal plant and its active ingredient, plumbagin. Redox Rep. 2004; 9: 219: 227.
6. da Silva Filho et al. (Hardness of low gold, 40%, content alloy Rev Odontol Univ Sao Paulo. 1988, 2: 127-130.
7. Zhang H. et al Lamiradosin, hepatitis C virus inhibitors from Lamium album. J Nat Prod. 2009; 72: 2158-2162.
8. Cheung R. et al. Compliance with anti-tuberculosis therapy. Eur J. Clin Pharmacol 1988; 35; 401-407.
9. Wang Y J et al. Diosginin, a plant-derived sapogenin, exhibits anti viral activity INVITRO against hepatitis C virus. J. Nat. Prod. 2011; 74; 580-584.
Structural Biology of Hepatitis C Virus, https://pubmed.ncbi.nlm.nih.gov/14752815/.
Binding-Site Identification and Genotypic Profiling of Hepatitis C Virus Polymerase Inhibitors, https://www.ncbi.nlm.nih.gov/pmc/articles/PMC1933266/.
Medicinal plants against hepatitis C virus, https://www.ncbi.nlm.nih.gov/pmc/articles/PMC3961971/.
Macrocyclic Hepatitis C Virus NS3/4A Protease Inhibitors: An Overview of Medicinal Chemistry, https://pubmed.ncbi.nlm.nih.gov/27160539/.
Journal of Hepatology, In Vitro Antiviral Activity of IDX320, A Novel and Potent Macrocyclic HCV Protease Inhibitor, https://www.journal-of-hepatology.eu/article/50168-8278(10)60770-2/pdf.
Safety and clinical effects of IDX320 in Hepatitis C infection, https://doi.org/10.1 186/ISRCTN44746369.
Idenix Pharmaceuticals Research and Development Update on HCV Programs, Phase 1/11: IDX320, an HCV protease inhibitor, http://www.natap.org/2010/HCV/072710_01.htm.
HCV NS3/4A serine protease in complex with 6570, https://www.rcsb.org/structure/4u01.
In Silico Identification and Evaluation of Leads for the Simultaneous Inhibition of Protease and Helicase Activities of HCV NS3/4A Protease Using Complex Based Pharmacophore Mapping and Virtual Screening, https://www.ncbi.nlm.nih.gov/pmc/articles/PMC3923879/.
Understanding the Structural and Energetic Basis of Inhibitor and Substrate Bound to the Full-Length NS3/4A: Insights From Molecular Dynamics Simulation, Binding Free Energy Calculation and Network Analysis, https://pubmed.ncbi.nlm.nih.gov/22833015/.
Full-length HCV NS3-4A protease-helicase in complex with a macrocyclic protease inhibitor, https://www.rcsb.org/structure/4a92.
A Macrocyclic HCV NS3/4A Protease Inhibitor Interacts with Protease and Helicase Residues in the Complex with its Full-Length Target, https://pubmed.ncbi.nlm.nih.gov/22160684/.
Simultaneously Targeting the NS3 Protease And Helicase Activities For More Effective Hepatitis C Virus Therapy, https://www.ncbi.nlm.nih.gov/pmc/articles/PMC4546510/.
A macrocyclic HCV NS3/4A protease inhibitor interacts with protease and helicase residues in the complex with its full-length target, https://www.ncbi.nlm.nih.gov/pmc/articles/PMC3248494/.
NS5B RNA Dependent RNA Polymerase Inhibitors: The Promising Approach to Treat Hepatitis C Virus Infections, https://pubmed.ncbi.nlm.nih.gov/20858218/.

The present general inventive concept may include a method for the treatment of a hepatitis disease, including administering to a subject in need thereof of an anti-pathogenic compound, such that the anti-pathogenic compound is derived from an herbal extract.

The herbal extract may be a glycol derivative.

The glycol derivative may be diethylene glycol dibenzoate.

The hepatitis disease may be hepatitis C.

The present general inventive concept may also include a method of strengthening a subject infected with a hepatitis disease, including administering to the subject in need thereof of an anti-pathogenic compound, such that the anti-pathogenic compound is derived from an herbal extract.

The herbal extract may be a glycol derivative.

The glycol derivative may be diethylene glycol dibenzoate.

The hepatitis disease may be caused by hepatitis C virus.

The anti-pathogenic compound may cause transversion and cross-links to a lipid-protein coat of the hepatitis C virus to inhibit the hepatitis C virus at entry, such that the hepatitis C virus is prevented from fusing with a plasma membrane of a cell.

The anti-pathogenic compound may prevent replication of the hepatitis C virus by inhibiting NS3 protease.

The anti-pathogenic compound may prevent capsomere assembly during a late-late stage of a life cycle of the hepatitis C virus.

The anti-pathogenic compound may boost an immune system of the subject.

The anti-pathogenic compound may boost the immune system by stimulating production of gamma interferon.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A method for the treatment of a hepatitis disease, comprising:
administering to a subject in need thereof of an anti-pathogenic compound,
such that the anti-pathogenic compound is derived from an herbal extract.

2. The method of claim 1, wherein the herbal extract is a glycol derivative.

3. The method of claim 2, wherein the glycol derivative is diethylene glycol dibenzoate.

4. The method of claim 1, wherein the hepatitis disease is hepatitis C.

5. A method of strengthening a subject infected with a hepatitis disease, comprising:
administering to the subject in need thereof of an anti-pathogenic compound,
such that the anti-pathogenic compound is derived from an herbal extract.

6. The method of claim 5, wherein the herbal extract is a glycol derivative.

7. The method of claim 6, wherein the glycol derivative is diethylene glycol dibenzoate

8. The method of claim 6, wherein the hepatitis disease is caused by hepatitis C virus.

9. The method of claim 8, wherein the anti-pathogenic compound causes transversion and cross-links to a lipid-protein coat of the hepatitis C virus to inhibit the hepatitis C virus at entry, such that the hepatitis C virus is prevented from fusing with a plasma membrane of a cell.

10. The method of claim 8, wherein the anti-pathogenic compound prevents replication of the hepatitis C virus by inhibiting NS3 protease.

11. The method of claim 8, wherein the anti-pathogenic compound prevents capsomere assembly during a late-late stage of a life cycle of the hepatitis C virus.

12. The method of claim 5, wherein the anti-pathogenic compound boosts an immune system of the subject.

13. The method of claim 12, wherein the anti-pathogenic compound boosts the immune system by stimulating production of gamma interferon.
